(19) United States
(12) Patent Application Publication
(10) Pub. No.: US 2013/0244265 A1
(43) Pub. Date: Sep. 19, 2013

(54) SECRETION OF RECOMBINANT POLYPEPTIDES IN THE EXTRACELLULAR MEDIUM OF DIATOMS

(71) Applicants: Alexandre Lejeune, La Chapelle Sur Erdre (FR); Rémy Michel, Nantes (FR); Jean-Paul Cadoret, Basse Goulaine (FR); Aude Carlier, Nantes (FR)

(72) Inventors: Alexandre Lejeune, La Chapelle Sur Erdre (FR); Rémy Michel, Nantes (FR); Jean-Paul Cadoret, Basse Goulaine (FR); Aude Carlier, Nantes (FR)

(73) Assignee: ALGENICS, Saint Herblain (FR)

(21) Appl. No.: 13/880,786

(22) PCT Filed: Oct. 20, 2011
(86) PCT No.: PCT/EP2011/005282

§ 371 Date: Jun. 6, 2013

(30) Foreign Application Priority Data
Oct. 20, 2010 (EP) 10013808.0

(51) Int. Cl. C12P 21/00

(52) U.S. Cl. CPC C12P 21/00
USPC 435/23; 435/257.2; 435/69.1

(57) ABSTRACT

A transformed diatom includes a nucleic acid sequence operatively linked to a promoter, wherein the nucleic acid sequence encodes an amino acid sequence including (i) an heterologous signal peptide and (ii) a polypeptide, the heterologous signal peptide leading to the secretion of the polypeptide in the extracellular medium of the transformed diatom; a method for producing a polypeptide which is secreted in the extracellular medium, the method including the steps of (i) culturing a transformed diatom, (ii) harvesting the extracellular medium of the culture and (iii) purifying the secreted polypeptide in the extracellular medium; and use of the transformed diatom for the secretion of a polypeptide in the extracellular medium.

### FIELD OF THE INVENTION

The present invention is directed to methods for producing recombinant proteins in diatoms, said polypeptides being secreted in the liquid culture medium.

### BACKGROUND OF THE INVENTION

The present invention relates to the production of recombinant proteins in diatoms. There is a high demand for these recombinant proteins in various domains such as biopharmaceuticals used in therapeutic applications or enzymes used as biocatalysts for industrial processes. As described by the international patent application WO 2009/101160, microalgae are an expression system of choice for the production of recombinant glycosylated proteins over alternative systems such as bacteria, yeast, fungi, plants or animals. Indeed, microalgae are able to perform complex glycosylation of interest. Microalgae present also the advantage of being cultivated in confined photobioreactors or conventional fermentors, therefore overcoming the problem of gene dissemination into the environment. In addition, microalgae cultures provide excellent yield in biomass in a short time and only requires synthetic sea water or fresh water, a total chemically defined media, as well as light or a carbon source for heterotrophic growing algae.

When producing recombinant proteins, one has to address the purification of them which is often tedious. However, this process can be greatly facilitated by the secretion of the protein in the culture broth. By reducing the number of steps to achieve suitable purity of the products, this leads to an improvement of the overall cost-effectiveness.

In eukaryotes, secreted proteins are translocated across the endoplasmic reticulum (ER) membrane, through the Golgi apparatus and subsequently released in the extracellular medium by secretory vesicles. The protein to be secreted is first produced with an amino-terminal located signal peptide which targets the polypeptide to the endosecretory pathway. This signal peptide is necessary to address the polypeptide to the endoplasmic reticulum and sufficient to lead to the secretion of the aforementioned protein to the extracellular media. During the translocation in the ER/Golgi, the signal peptide is cleaved and the protein is being matured (undergo post translational modifications). It allows the delivery in the culture media of complex mature proteins.

Traditionally, signal peptides are viewed as being functional across species based on their shared characteristics in eukaryotes. For example, human or plant signal sequences can successfully lead to the secretion of recombinant proteins when used in the yeast Pichia pastoris. In plant, studies revealed that murine signal peptide sequences can also be functional. Nevertheless, data in the literature proved that this assumption could not be further from the truth. For example, 4 proteins (VSG 117, VSG MVAT7, VSG 221 and BiP) from Trypanosoma brucei and one protein (gp63) from Leishmania sp. harboring signal peptide were not translocated into dog pancreatic microsomes used to mimic the passage into the ER membrane (Al-Qahtani et al., 1998). Similarly, signal peptide of the carboxypeptidase Y from the yeast Saccharomyces cerevisiae did not led to the translocation into the ER of this recombinant protein when expressed in the mammalian COS-1 cells (Bird et al., 1987).

In the prior art, the international patent application WO 2009/101160 describes the expression of glycosylated proteins in microalgae and furthermore the analysis of the glycosylation of said proteins from crude extracts of microalgae. However, said international patent application does not specifically describe nor suggest the use of a heterologous signal peptide, and especially a mammal signal peptide, leading directly to the secretion of polypeptides in the extracellular medium of microalgae, no more than the secretion into the extracellular medium of microalgae of the glycoproteins expressed in said microalgae. On the contrary, the analysis of the glycoproteins from crude extracts as described in the international patent application WO 2009/101160 indicates that said glycoproteins are intended to be found in the microalgae and not in their extracellular medium.

Furthermore, the prior art does not describe nor suggest the use of a heterologous signal peptide, and especially a mammal signal peptide, leading to the secretion of proteins in the extracellular medium of microalgae. To date, no study has been realized to test whether an exogenous signal peptide could lead to the secretion of recombinant proteins in microalgae, and especially in diatoms. Indeed, inferring the secretion machinery based on prior knowledge is hampered by the phylogenetic distance of these microalgae which belong to a eukaryotic phylum faraway from other organisms such as animals. As a member of the eukaryotic lineage Chromalveolates, diatoms are evolutionarily distinct from the plantae, the lineage containing land plants, green and red algae and the opisthokonta containing fungi and metazoa as shown in FIG. 1 (Keeling et al., 2005). A broad gene analysis has revealed major differences in the diatom P. tricornutum, when compared to plantae and opisthokonta. Thus, amongst the 3710 gene families identified in P. tricornutum, nearly 40% could not be found in plantae and/or opisthokonta (Bowler et al., 2008).

### SUMMARY OF THE INVENTION

In a first aspect, the present invention provides a transformed diatom comprising a nucleic acid sequence operatively linked to a promoter, wherein said nucleic acid sequence encodes an amino acid sequence comprising:
(i) an heterologous signal peptide; and
(ii) a polypeptide,
said heterologous signal peptide leading to the secretion of said polypeptide in the extracellular medium of said transformed diatom.

In a preferred embodiment, the transformed diatom is selected from the group comprising Bacillariophyceae diatoms.

In a most preferred embodiment, the transformed diatom is Phaeodactylum tricornutum.

In a second aspect, the present invention relates to a method for producing a polypeptide which is secreted in the extracellular medium, said method comprising the steps of:
(i) culturing a transformed diatom as defined previously;
(ii) harvesting the extracellular medium of said culture; and
(iii) purifying the secreted polypeptide in said extracellular medium.

In a third aspect, the present invention refers to the use of a transformed diatom for the secretion of a polypeptide in the extracellular medium.

### BRIEF DESCRIPTION OF DRAWINGS

FIG. 1. Diatoms Phylogeny

FIG. 2. Normalized secreted Luciferase activity of Phaeodactylum tricornutum transformants.

FIG. 3. Detection of secreted Gaussia Luciferase by Western Blot.

FIG. 4. Detection of secreted Erythropoietin by Western Blot.

FIG. 5. Detection of secreted chimeric eGFP by Western Blot.

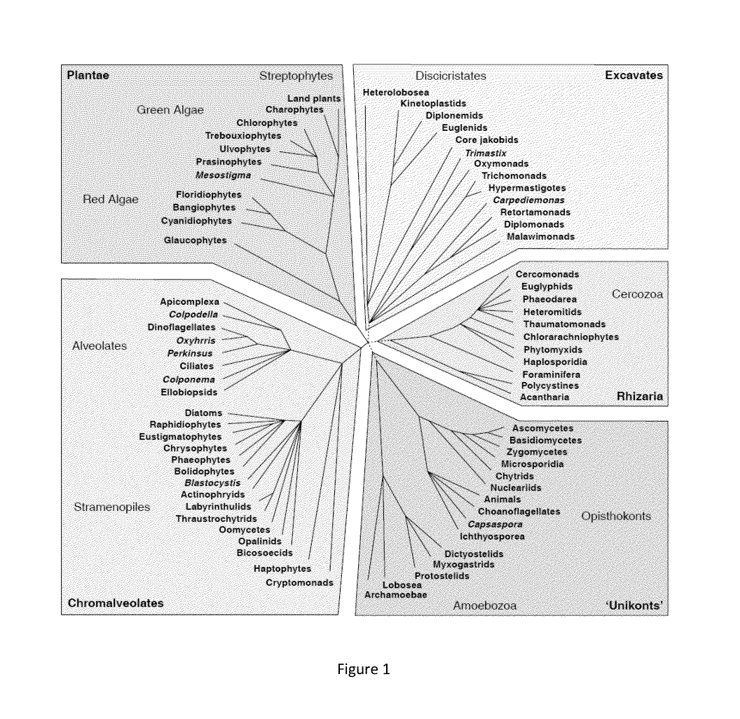

### DETAILED DESCRIPTION OF THE INVENTION

The invention aims to provide a new system for producing recombinant polypeptides in a diatom, said polypeptides being secreted in the liquid culture medium.

The applicant surprisingly found that transformed diatoms were capable of producing and secreting a polypeptide in their extracellular media, when being transformed with a sequence encoding a polypeptide and a heterologous signal peptide.

An object of the invention is a transformed diatom comprising a nucleic acid sequence operatively linked to a promoter, wherein said nucleic acid sequence encodes an amino acid sequence comprising:
(i) an heterologous signal peptide; and
(ii) a polypeptide,
said heterologous signal peptide leading to the secretion of said polypeptide in the extracellular medium of said transformed diatom.

The term “nucleic acid sequence” used herein refers to DNA sequences (e.g., cDNA or genomic or synthetic DNA) and RNA sequences (e. g., mRNA or synthetic RNA), as well as analogs of DNA or RNA containing non-natural nucleotide analogs, non-native internucleoside bonds, or both. Preferably, said nucleic acid sequence is a DNA sequence. The nucleic acid can be in any topological conformation, like linear or circular.

“Operatively linked” promoter refers to a linkage in which the promoter is contiguous with the gene of interest to control the expression of said gene.

Examples of promoter that drives expression of a polypeptide in transformed diatoms include, but are not restricted to, nuclear promoters such as fcpA and fcpB from Phaeodactylum tricornutum (Zavlaskaïa et al. (2000) Transformation of the diatom Phaeodactylum tricornutum (Bacillariophyceae) with a variety of selectable marker and reporter genes. J. Phycol. 36, 379-386).

Transformation of diatoms can be carried out by conventional methods such as microparticles bombardment, electroporation, glass beads, polyethylene glycol (PEG). Such a protocol is disclosed in the examples.

In an embodiment of the invention, nucleotide sequences may be introduced into diatoms via a plasmid, virus sequences, double or simple strand DNA, circular or linear DNA.

In another embodiment of the invention, it is generally desirable to include into each nucleotide sequences or vectors at least one selectable marker to allow selection of diatoms that have been stably transformed. Examples of such markers are antibiotic resistant genes such as sh ble gene enabling resistance to zeocin, nat or sat-1 genes enabling resistance to nourseothricin.

After transformation of diatoms, transformants producing the desired proteins secreted in the culture media are selected. Selection can be carried out by one or more conventional methods comprising: enzyme-linked immunosorbent assay (ELISA), mass spectroscopy such as MALDI-TOF-MS, ESI-MS chromatography, spectrophotometer, fluorimeter, immunocytochemistry by exposing cells to an antibody having a specific affinity for the desired protein.

The term “polypeptide” as used herein refers to an amino acid sequence comprising amino acids which are linked by peptide bonds. A polypeptide may be monomeric or polymeric. Furthermore, a polypeptide may comprise a number of different domains each of which has one or more distinct activities.

The term “peptide” as used herein refers to an amino acid sequence that is typically less than 50 amino acids long and more typically less than 30 amino acids long.

The term “signal peptide” as used herein refers to an amino acid sequence which is generally located at the amino terminal end of the amino acid sequence of a polypeptide. The signal peptide mediates the translocation of said polypeptide through the secretion pathway and leads to the secretion of said polypeptide in the extracellular medium.

As used herein, the term “secretion pathway” refers to the process used by a cell to secrete proteins out of the intracellular compartment. Such pathway comprises a step of translocation of a polypeptide across the endoplasmic reticulum membrane, followed by the transport of the polypeptide in the Golgi apparatus, said polypeptide being subsequently released in the extracellular medium of the cell by secretory vesicles. Post-translational modifications necessary to obtain mature proteins, such as glycosylation or disulfide bonds formation, are operated on proteins during said secretion pathway.

Preferably, the signal peptide leading to the secretion of the polypeptide in the extracellular medium is located at its amino-terminal end.

This signal peptide is typically 15-30 amino acids long, and presents a 3 domains structure (von Heijne G. (1990) The signal Peptide, J Membr Biol, 115:195-201; Emanuelsson O. et al (2007) Locating proteins in the cell using TargetP, SignalP and related tools. Nat Protoc 2:953-971), which are as follows:
(i) an N-terminal region (n-region) containing positively charged amino acids, such as Arginine (R), Histidine (H) or Lysine (K);
(ii) a central hydrophobic region (h-region) of at least 6 amino acids containing hydrophobic amino acids such as Alanine (A), Cysteine (C), Glycine (G), Isoleucine (I), Leucine (L), Methionine (M), Phenylalanine (F), Proline (P), Tryptophan (W) or Valine (V); and
(iii) a C-terminal region (c-region) of polar uncharged amino acids such as Asparagine (R), Glutamine (Q), Serine (S), Threonine (T) or Tyrosine (Y). Said C-region often contains a helix-breaking proline or glycine that helps define a cleavage site. Small uncharged residues in positions −3 and −1 (defined as the number of residue before the cleavage site) are usually requires for an efficient cleavage by signal peptidase following the translocation across the endoplasmic reticulum membrane (von Heijne G. (1990) The signal Peptide, J Membr Biol 115:195-201; Vernet K., Schatz G. (1988) Protein translocation across membranes, Science, 241:1307-1313).

A person skilled in the art is able to simply identify a signal peptide in an amino acid sequence, for example by using the SignalP 3.0 Server (accessible on line at http://www.cbs.dtu.dk/services/SignalP/) which predicts the presence and location of signal peptide cleavage sites in amino acid sequences from different organisms by using two different models: the Neural networks and the Hidden Markov models (Emanuelsson O. et al (2007) Locating proteins in the cell using TargetP, SignalP and related tools. Nat Protoc 2:953-971).

The term “heterologous”, with reference to a signal peptide or to a polypeptide, means an amino acid sequence which does not exist in the corresponding diatom before its transformation. It is intended that the term encompasses proteins that are encoded by wild-type genes, mutated genes, and/or synthetic genes.

In a preferred embodiment, the polypeptide secreted in the extracellular medium of transformed diatoms according to the invention is a heterologous polypeptide.

Advantageously, the heterologous signal peptide used herein corresponds to the signal peptide of said heterologous polypeptide, said signal peptide leading to the secretion of said heterologous polypeptide in the extracellular medium of the cell from which it is originate. An example of such embodiment is disclosed in the examples, wherein the signal peptide leading to the secretion of Gaussia princeps luciferase in P. tricornutum is its native signal peptide.

In a still preferred embodiment, said heterologous polypeptide which is secreted in the extracellular medium of the transformed diatom according to the invention can be of animal origin. Preferably, said polypeptide is of mammalian origin. Most preferably, said polypeptide is of human origin. Examples of such embodiment in the present invention include the murine erythropoietin and the human interleukin-2.

In another preferred embodiment, the polypeptide to be secreted in the extracellular medium of the transformed diatoms of the invention is a protein of therapeutic interest selected in the group comprising antibodies and their fragments, erythropoietin, cytokines such as interferons, coagulation factors, hormones, beta-glucocerebrosidase, pentraxin-3, anti-TNFs, α-glucosidase acide, α-L-iduronidase and derivatives thereof.

An antibody is an immunoglobulin molecule corresponding to a tetramer comprising four polypeptide chains, two identical heavy (H) chains (about 50-70 kDa when full length) and two identical light (L) chains (about 25 kDa when full length) inter-connected by disulfide bonds. Light chains are classified as kappa and lambda. Heavy chains are classified as gamma, mu, alpha, delta, or epsilon, and define the antibody's isotype as IgG, IgM, IgA, IgD, and IgE, respectively. Each heavy chain is comprised of an amino-terminal heavy chain variable region (abbreviated herein as HCVR) and a heavy chain constant region. The heavy chain constant region is comprised of three domains (CH1, CH2, and CH3) for IgG, IgD, and IgA; and 4 domains (CH1, CH2, CH3, and CH4) for IgM and IgE. Each light chain is comprised of an amino-terminal light chain variable region (abbreviated herein as LCVR) and a light chain constant region. The light chain constant region is comprised of one domain, CL. The HCVR and LCVR regions can be further subdivided into regions of hypervariability, termed complementarity determining regions (CDRs), interspersed with regions that are more conserved, termed framework regions (FR). Each HCVR and LCVR is composed of three CDRs and four FRs, arranged from amino-terminus to carboxy-terminus in the following order: FR1, CDR1, FR2, CDR2, FR3, CDR3, FR4. The assignment of amino acids to each domain is in accordance with well-known conventions. The functional ability of the antibody to bind a particular antigen depends on the variable regions of each light/heavy chain pair, and is largely determined by the CDRs.

The term “antibody”, as used herein, refers to a monoclonal antibody per se. A monoclonal antibody can be a human antibody, chimeric antibody and/or humanized antibody.

The term “antibody fragments” as used herein refers to antibody fragments that bind to the particular antigens of said antibody. For example, antibody fragments capable of binding to particular antigens include Fab (e.g., by papain digestion), Fab′ (e.g., by pepsin digestion and partial reduction) and F(ab′)2 (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments, are encompassed by the invention.

Such fragments can be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. For example, a combination gene encoding a F(ab′)2 heavy chain portion can be designed to include DNA sequences encoding the CH₁ domain and/or hinge region of the heavy chain. The various portions of antibodies can be joined together chemically by conventional techniques, or can be prepared as a contiguous protein using genetic engineering techniques.

The term “Cytokines” refers to signaling proteins which are released by specific cells of the immune system to carry a signal to other cells in order to alter their function. Cytokines are immunomodulating agents and are extensively used in cellular communication. The term cytokines encompasses a wide range of polypeptide regulators, such as interferons, interleukins, chemokins or Tumor Necrosis Factor.

The term “Coagulation factors” refers to the plasma proteins which interact with platelets in a complex cascade of enzyme-catalyzed reactions, leading to the formation of fibrin for the initiation of a blood clot in the blood coagulation process. Coagulation factors, at the number of 13, are generally serine proteases, but also comprise glycoproteins (Factors VIII and V) or others types of enzyme, such as transglutaminase (Factor XIII).

The term “Hormones” refers to chemical messengers secreted by specific cells in the plasma or the lymph to produce their effects on other cells of the organism at a distance from their production sites. Most hormones initiate a cellular response by initially combining with either a specific intracellular or cell membrane associated receptor protein. Common known hormones are, for example, insulin for the regulation of energy and glucose in the organism, or the Growth Hormone which stimulates growth and cell reproduction and regeneration.

As used herein, the term “derivative” refers to a polypeptide having a percentage of identity of at least 90% with the complete amino acid sequence of any of the protein of therapeutic interest disclosed previously and having the same activity.

Preferably, a derivative has a percentage of identity of at least 95% with said amino acid sequence, and preferably of at least 99% with said amino acid sequence.

As used herein, “percentage of identity” between two amino acids sequences, means the percentage of identical amino-acids, between the two sequences to be compared, obtained with the best alignment of said sequences, this percentage being purely statistical and the differences between these two sequences being randomly spread over the amino acids sequences. As used herein, “best alignment” or “optimal alignment”, means the alignment for which the determined percentage of identity (see below) is the highest. Sequences comparison between two amino acids sequences are usually realized by comparing these sequences that have been previously aligned according to the best alignment; this comparison is realized on segments of comparison in order to identify and compare the local regions of similarity. The best sequences alignment to perform comparison can be realized by using computer softwares using algorithms such as GAP, BESTFIT, BLAST P, BLAST N, FASTA, TFASTA in the Wisconsin Genetics software Package, Genetics Computer Group, 575 Science Dr., Madison, Wis. USA. To get the best local alignment, one can preferably used BLAST software, with the BLOSUM 62 matrix, preferably the PAM 30 matrix. The identity percentage between two sequences of amino acids is determined by comparing these two sequences optimally aligned, the amino acids sequences being able to comprise additions or deletions in respect to the reference sequence in order to get the optimal alignment between these two sequences. The percentage of identity is calculated by determining the number of identical position between these two sequences, and dividing this number by the total number of compared positions, and by multiplying the result obtained by 100 to get the percentage of identity between these two sequences.

In a most preferred embodiment of the invention, the nucleic acid sequence encoding an amino acid sequence comprising (i) an heterologous signal peptide and (ii) a polypeptide, said heterologous signal peptide leading to the secretion of said polypeptide in the extracellular medium of diatoms, are selected in the group comprising the sequences disclosed in Table I.

TABLE I

| | | | | |
|---|---|---|---|---|
| | Accession | | Accession | |
| | numbers | CDS SEQ | number | |
| PROTEIN | (CDS) | ID N^{o} | (Protein) | Comments |

Interferons

| | | | |
|---|---|---|---|
| Interferon β1 | CCDS6495 | SEQ ID N^{o} 1 | NP_002167 |
| Interferon β2 | CCDS6506 | SEQ ID N^{o} 2 | NP_000596 |

Interleukins

| | | | |
|---|---|---|---|
| IL-11 | CCDS12923 | SEQ ID N^{o} 3 | NP_000632 |
| IL-6 = Interferon β2 | CCDS5375 | SEQ ID N^{o} 5 | NP_000591 |
| IL-21 | CCDS3727 | SEQ ID N^{o} 6 | NP_068575 |

Hormones

| | | | | |
|---|---|---|---|---|
| Insulin | J00265 | SEQ ID N^{o} 7 | AAA59172 | |
| Preproglucagon | V01515 | SEQ ID N^{o} 8 | CAA24759 | |
| Variants | | | | |
| EPO | CCDS5705 | SEQ ID N^{o} 9 | NP_000790 | |
| Growth hormone | CCDS11653 | SEQ ID N^{o} 10 | NP_000506 | isoform 1 |
| | CCDS45760 | SEQ ID N^{o} 11 | NP_072053 | isoform 2 |
| | CCDS11654 | SEQ ID N^{o} 12 | NP_072054 | isoform 3 |
| | CCDS42371 | SEQ ID N^{o} 13 | NP_072055 | isoform 4 |
| | NM_022562 | SEQ ID N^{o} 14 | NP_072056 | isoform 5 |
| GM-CSF(colony | CCDS4150 | SEQ ID N^{o} 15 | NP_000749 | |
| stimulating factor 2 | | | | |
| granulocyte- | | | | |
| macrophage) | | | | |
| G-CSF (Granulocyte- | CCDS11357 | SEQ ID N^{o} 16 | NP 000750 | isoform a |
| Colony stimulating | | | | |
| Factor 3 | | | | |
| Follicle stimulating | CCDS5007 | SEQ ID N^{o} 17 | NP_000726 | subunit alpha |
| hormone | CCDS7868 | SEQ ID N^{o} 18 | NP_000501 | subunit beta |
| Chorionic gonadotropin | CCDS5007 | SEQ ID N^{o} 17 | NP_000726 | subunit alpha |
| | CCDS12749 | SEQ ID N^{o} 19 | NP_000728 | subunit beta |
| Thyroid stimulating | CCDS5007 | SEQ ID N^{o} 17 | NP_000726 | subunit alpha |
| hormone (Thyrogen) | CCDS880 | SEQ ID N^{o} 20 | NP_000540 | subunit beta |
| Luteinizing hormone | CCDS5007 | SEQ ID N^{o} 17 | NP_000726 | subunit alpha |
| | CCDS12748 | SEQ ID N^{o} 21 | NP_000885 | subunit beta |

Coagulation factors

| | | | | |
|---|---|---|---|---|
| Factor II = thrombin | CCDS31476 | SEQ ID N^{o} 22 | NP_000497 | |
| Factor VII | J02933 | SEQ ID N^{o} 23 | AAA51983 | |
| Factor VIII | K01740 | SEQ ID N^{o} 24 | AAA52484 | |
| Factor IX | J00136 | SEQ ID N^{o} 25 | AAA98726 | |
| Tissue plasminogen | CCDS6127 | SEQ ID N^{o} 26 | NP_127509 | isoform 3 |
| activator | CCDS6126 | SEQ ID N^{o} 27 | NP_000921 | isoform 1 |
| Protein C | CCDS2145 | SEQ ID N^{o} 28 | NP_000303 | |

Lysosomal enzymes

| | | | |
|---|---|---|---|
| β-glucocerebrosidase = | CCDS1102 | SEQ ID N^{o} 29 | NP_000148 |
| β-glucosidase acid | | | |
| α-Galactosidase A | CCDS14484 | SEQ ID N^{o} 30 | NP_000160 |
| Alglucosidase = | CCDS32760 | SEQ ID N^{o} 31 | NP_000143 |
| α-glucosidase acid | | | |

Other proteins

| | | | | |
|---|---|---|---|---|
| Bone morphogenetic | CCDS13455 | SEQ ID N^{o} 32 | NP_001710 | |
| protein 7 = | | | | |
| osteogenic protein-1 | | | | |
| Bone morphogenetic | CCDS13099 | SEQ ID N^{o} 33 | NP_001191 | |
| protein 2 | | | | |
| α-L-iduronidase | CCDS3343 | SEQ ID N^{o} 34 | NP_000194 | |
| Pancreatic lipase | CCDS7594 | SEQ ID N^{o} 35 | NP_000927 | |
| Pancreatic amylases | CCDS783 | SEQ ID N^{o} 36 | NP_000690 | α-2A-amylase |
| | CCDS782 | SEQ ID N^{o} 37 | NP_066188 | α-2B-amylase |
| Gastric lipase | CCDS7389 | SEQ ID N^{o} 38 | NP_004181 | |
| Albumin | CCDS3555 | SEQ ID N^{o} 39 | NP_000468 | |

Antibodies

| | | | | |
|---|---|---|---|---|
| Immunoglobulin heavy | AJ294730 | SEQ ID N^{o} 40 | CAC20454 | Gamma 1 |
| chain constant region | AJ294733 | SEQ ID N^{o} 41 | CAC20457 | Gamma 4 |
| gamma | | | | |
| Immunoglobulin | M26995 | SEQ ID N^{o} 42 | AAA59127 | |
| Variable Heavy Chain | | | | |
| Immunoglobulin Kappa | AJ010442 | SEQ ID N^{o} 43 | CAA09181 | |
| light Chain (VL + CL) | | | | |

In another preferred embodiment, the polypeptide to be secreted in the extracellular medium of the transformed diatom of the invention is a protein allowing modifications of said diatom to improve its industrial application. Examples of such embodiment include the secretion by microalgae of enzymes in the extracellular media to modify its own cell wall in order to improve biodegradability and therefore biomass conversion efficiency for applications such as biofuels. Enzymes to be produced for hydrolysis of microalgal cell wall oligosaccharides into soluble sugars include, but are not limited to, mannosidases or galactosidases. In another example of such embodiment, enzymes secreted in the media allow the modification of cell wall to enhance adsorption ability of microalgae on solid support. Applications of such technology include immobilization of microalgae for used as biocatalyst, biosensor or in bioremediation processes.

In another embodiment of this invention, polypeptides to be produced in the extracellular media are ligninolytic enzymes used in green chemistry. Examples of these enzymes include, but are not limited to, lignin peroxidases, manganese-dependant peroxidases and laccases. By improving the biodegradability of wood material, these enzymes have biotechnological applications in biopulping and biofuel production from plant origin. These enzymes can also be used to treat industrial waste such as polluted water containing toxic dyes from the textile industry.

Another embodiment of this invention is the genetic engineering of optimal biomaterials based on microalgal carbohydrate polymers. An example of enzymes to be secreted in the media for such applications includes peroxidases such as horseradish peroxidase allowing the cross-linking of tyramine-conjugated polymers to form hydrogel. In another example of this application, the enzyme to be secreted in the media is a transglutaminases to perform cross-linking of proteins of interest onto the sugar backbone of carbohydrate polymers.

The term “enzyme”, when used herein refers to a molecule having at least one enzymatic activity, and includes full-length enzymes, catalytically active fragments, chimerics, complexes, and the like. A “catalytically active fragment” of an enzyme refers to a polypeptide having a detectable level of functional (enzymatic) activity.

Host cells used herein for the secretion of a polypeptide in the extracellular medium are aquatic photosynthetic microorganism which belongs to Bacillariophyceae also known as Diatoms.

In a most preferred embodiment, the diatom is Phaeodactylum tricornutum.

In another embodiment of the invention, diatoms used herein for the secretion of polypeptides in the extracellular medium further express an N-acetylglucosaminyltransferase (GnT I, GnT II, GnT III, GnT IV, GnT V or GnT VI), a mannosidase II and a fucosyltransferase, galactosyltransferase (GalT) or sialyltransferases (ST), to secrete glycosylated polypeptides. Glycosylation is dependent on the endogenous machinery present in the host cell chosen for producing and secreting glycosylated polypeptides. Diatoms are capable of producing such glycosylated polypeptides in high yield via their endogenous N-glycosylation machinery.

Another object of the invention is a method for producing a polypeptide which is secreted in the extracellular medium, said method comprising the steps of:
(i) culturing a transformed diatom as described above;
(ii) harvesting the extracellular medium of said culture; and
(iii) purifying the polypeptide, which is secreted in said extracellular medium.

In another embodiment of the invention, the method of producing a polypeptide which is secreted in the extracellular medium of diatoms comprises a former step of transforming said diatoms with a nucleic acid sequence operatively linked to a promoter, wherein said nucleic acid sequence encodes an amino acid sequence comprising an heterologous signal peptide and a polypeptide, said heterologous signal peptide leading to the secretion of said polypeptide in the extracellular medium of said transformed diatom.

In another embodiment of the invention, the method of producing secreted polypeptide in the extracellular medium of transformed diatoms further comprises a step (iv) of determining the glycosylation pattern of said polypeptide.

Preliminary information about N-glycosylation of the recombinant polypeptide secreted in the extracellular medium can be obtained by affino- and immunoblotting analysis using specific probes such as lectins (CON A; ECA; SNA; MAA . . . ) and specific N-glycans antibodies (anti-1,2-xylose; anti-1,3-fucose; anti-Neu5Gc, anti-Lewis . . . ). To investigate the detailed N-glycan profile of recombinant polypeptide, N-linked oligosaccharides is then released from the polypeptide in a non specific manner using enzymatic digestion or chemical treatment. The resulting mixture of reducing oligosaccharides can be profiled by HPLC and/or mass spectrometry approaches (ESI-MS-MS and MALDI-TOF essentially). These strategies, coupled to exoglycosidase digestion, enable N-glycan identification and quantification (Séveno et al., 2008, Plant N-glycan profiling of minute amounts of material, Anal. Biochem., vol. 379 (1), p: 66-72; Stadlmann et al., 2008, Analysis of immunoglobulin glycosylation by LC-ESI-MS of glycopeptides and oligosaccharides. Proteomics, vol. 8, p: 2858-2871).

In a preferred embodiment, the method of producing a polypeptide secreted in the extracellular medium of diatoms leads to the secretion of at least 25%, 50%, 75% or 90% of the polypeptide expressed in said diatoms.

Secretion efficiency can be assessed using pulse-chase experiments with radiolabeled amino acids, as described by Jensen et al. (2000), except that media are replaced by those used to grow diatoms. The protein to study is then immunoprecipitated on both intracellular and extracellular fractions and subjected to SDS-PAGE electrophoresis and quantified using the phosphor-imaging technology.

The percentage of secretion for any given time can be calculated as follow:

QSecreted+Qinternal=100% of expressed polypeptides

% secreted=(Qsecreted×100%)/(Qsecreted+Qinternal)

Said formula can be merely explained as following:
quantity of the polypeptide of interest in the extracellular medium of transformed diatoms (Qsecreted);
quantity of said polypeptide in the intracellular medium of transformed diatoms (Qinternal)
Additioning both quantities as determined precedently to obtain the total quantity of produced polypeptides by the transformed diatoms, such quantity being equivalent to 100% (100% of expressed polypeptides)
Multiplying the amount of secreted polypeptides (Qsecreted) by 100%, and dividing the result by the total of polypeptides expressed by the transformed diatoms (Qsecreted+Qinternal) to obtain the percentage of polypeptides secreted in the extracellular medium of said diatoms (% secreted).

Another object of the invention is the use of a transformed diatom as previously described for the secretion of a polypeptide in the extracellular medium.

In the following, the invention is described in more detail with reference to methods. Yet, no limitation of the invention is intended by the details of the examples. Rather, the invention pertains to any embodiment which comprises details which are not explicitly mentioned in the examples herein, but which the skilled person finds without undue effort.

### EXAMPLES

### Example 1

### Secretion of Gaussia princeps Luciferase in the Culture Medium of Transformed Phaeodactylum tricornutum

To test the functionality of an exogenous signal peptide, Phaeodactylum tricornutum (P. Tricomutum) was transformed with a plasmid containing Gaussia princeps luciferase (GLuc) coding sequence. This luciferase is responsible for the bioluminescent reaction of the marine copepod Gaussia princeps. Its amino terminal extremity carried a signal peptide leading to the natural secretion of the enzyme in the extracellular medium. The whole native GLuc sequence including the signal peptide from G. princeps was used to transform P. tricornutum. As a control, P. tricornutum was also transformed with the GLuc sequence lacking the signal peptide as determined using SignalP.

a) Standard Culture Conditions of Phaeodactylum tricornutum

Strains used in this work were Phaeodactylum tricornutum. Diatoms were grown at 20° C. under continuous illumination (280-350 μmol photons.m⁻².s⁻¹), in natural coastal seawater sterilized by 0.22 μm filtration. This seawater is enriched with nutritive Conway media (Walne, 1966) with addition of silica (40 mg/L of sodium metasilicate). For large volume (from 2 liters to 300 liters) cultures were aerated with a 2% CO₂/air mixture to maintain the pH in a range of 7.5-8.1.

For genetic transformation, diatoms were spread on gelose containing 1% of agar. After concentration by centrifugation, the diatoms were spread on petri dishes sealed and incubated at 20° C. under constant illumination. Concentration of cultures was estimated on Mallassez counting cells after fixation of the microalgae with a Lugol's solution.

b) Expression Constructs for GLuc

The cloning vector pPHA-T1 built by Zavlaskaïa et al. (2000) includes sequences of P. tricornutum promoters fcpA and fcpB (fucoxanthin-chlorophyll a/c-binding proteins A and B) and the terminator of fcpA. It contains a selection cassette with the gene she ble and a MCS flanking the fcpA promoter. Gaussia luciferase is encoded by a 558 pb sequence (SEQ ID N°44). The full length Gaussia luciferase coding sequence was synthesized with the addition of EcoRI and HindIII restriction sites flanking the 5′ and 3′ ends respectively. As a control, a Gaussia luciferase coding sequence lacking the signal peptide was also synthetized (SEQ ID N°45) with EcoRI and HindIII restriction sites at both ends. After digestion by EcoRI and HindIII, both inserts were introduced into pPHA-T1 vectors. A vector lacking the luciferase coding sequence was used as control.

c) Genetic Transformation

The transformation was carried out by particles bombardment using the BIORAD PDS-1000/He apparatus modified by Thomas J L. et al. (2001) A helium burst biolistic device adapted to penetrate fragile insect tissues, Journal of Insect Science, 1-9).

Cultures of diatoms (P. tricornutum) in exponential growth phase were concentrated by centrifugation (10 minutes, 2150 g, 20° C.), diluted in sterile seawater, and spread on geloses at 10⁸ cells per dish. The microcarriers were gold particles (diameter 0.6 μm). Microcarriers were prepared according to the protocol of the supplier (BIORAD). Parameters used for shooting were the following:
use of the long nozzle,
use of the stopping ring with the largest hole,
15 cm between the stopping ring and the target (diatoms cells),
precipitation of the DNA with 1.25 M CaCl₂ and 20 mM spermidine,
a ratio of 1.25 μg DNA for 0.75 mg gold particles per shot,
rupture disk of 900 psi with a distance of escape of 0.2 cm,
a vacuum of 30H g

Diatoms were incubated 24 hours before the addition of the antibiotic zeocin (100 μg/ml) and were then maintained at 20° C. under constant illumination. After 1-2 weeks of incubation of the plates, individual clones were picked from the plates and inoculated into liquid medium containing zeocin (100 μg/ml).

d) Microalgae DNA Extraction

Cells (5.10⁸) transformed by the vector bearing the full-length GLuc, GLuc lacking the signal peptide or control plasmid were pelleted by centrifugation (2150 g, 15 minutes, 4° C.). Microalgae cells were incubated overnight at 4° C. with 4 mL of TE NaCl 1× buffer (Tris-HCL 0.1 M, EDTA 0.05 M, NaCl 0.1 M, pH 8). 1% SDS, 1% Sarkosyl and 0.4 mg.mL⁻¹ of proteinase K were then added to the sample, followed by an incubation at 40° C. for 90 minutes. A first phenol-chloroform isoamyl alcohol extraction was carried out to extract an aqueous phase comprising the nucleic acids. RNA presents in the sample was eliminated by an hour incubation at 60° C. in the presence of RNase (1 μg.mL⁻¹). A second phenol-chloroform extraction was carried out, followed by a precipitation a precipitation with ethanol. Finally, the pellet was dried and solubilised into 200 μL of ultrapure sterile water. Quantification of DNA was carried out by spectrophotometry (260 nm) and analysed by agarose gel electrophoresis.

e) Polymerase Chain Reaction (PCR) Analysis

The incorporation of the heterologous full-length GLuc and Gluc lacking the signal peptide in the genome of Phaeodactylum tricornutum was assessed by PCR analysis. The sequence of primers used for the amplification of GLuc transformed cells were 5′-CATTGTAGCTGTAGCTAGC-3′ (SEQ ID N°46) and 5′-TTAATCACCACCGGCAC-3′(SEQ ID N°47). The PCR reaction was carried out in a final volume of 50 μl consisting of 1× PCR buffer, 0.2 mM of each dNTP, 5 μM of each primer, 20 ng of template DNA and 1.25 U of Taq DNA polymerase (Taq DNA polymerase, ROCHE). Thirty cycles were conducted for amplification of template DNA. Initial denaturation was performed at 94° C. for 4 min. Each subsequent cycle consisted of a 94° C. (1 min) melting step, a 55° C. (1 min) annealing step, and a 72° C. (1 min) extension. Samples obtained after the PCR reaction were run on agarose gel (1%) stained with ethidium bromide.

Results revealed a single band at 478 bp for cells transformed with the constructs carrying the full-length GLuc or Gluc lacking its signal peptide (data not shown). No band was detected in cells transformed with the control vector. This result validates the incorporation of exogenous gene in the genome of Phaeodactylum tricornutum.

f) Luciferase Activity

GLuc catalyzes the oxidative decarboxylation of coelenterazine to produce the excited state of coelenteramide, which upon relaxation to the ground state emits light. This enzymatic property was used to test the presence and functionality of GLuc in P. tricornutum.

The luciferase activity was measured in the culture medium of transformants harboring the full-length GLuc (92 cell lines), GLuc lacking its signal peptide (90 cell lines) as well as cells transformed with the control vector (96 cell lines). A 96 wells microplate luminometer with automated substrate injection was used (Victor™ X3, Perkin Elmer). The coelenterazine substrate (Luxinnovate) was resuspended in acidic ethanol at a concentration of 5 mg/mL and this stock solution was stored at −80° C. Prior to measurements, a working solution of substrate was prepared by diluting the stock solution in distillated water (1:300). This solution was kept at room temperature for 20 minutes before the start of the experiment. P. tricornutum transformed with the full-length Gaussia luciferase or lacking the signal peptide as well as wild-type cells were grown in 96 wells microplate and centrifuged (10 minutes, 2150 g, 20° C.) at exponential phase of growth. Forty μL of culture supernatant was then mixed with 40 μL of the coelenterazine working solution using automated injection and shaking. Light emission was recorded for 10 seconds.

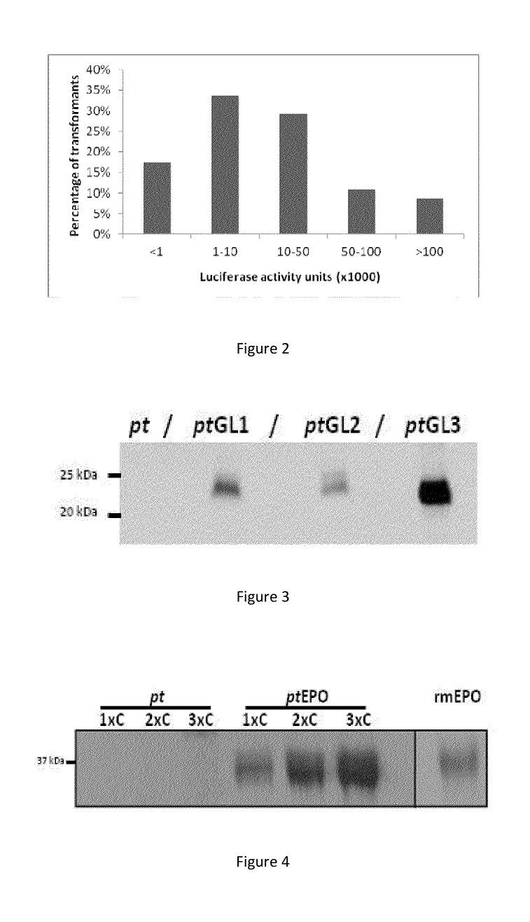

Cells transformed with the full-length GLuc sequence were classified into 5 groups depending on their luciferase activity (FIG. 2). Variable levels of luciferase activity were detected in the full-length GLuc transformants tested ranging from signals corresponding to the background (i.e. <1000 light units) to signals above 1.10⁶ light units. This wide distribution is typically observed for non-homologous transformation of the nuclear genome. Indeed, the number of transgene copies inserted in the nuclear genome and/or the location in the genome can vary between clones resulting in variable level of transgene expression. No luciferase activity above the background was detected for cells transformed with GLuc lacking the signal peptide or control cells (data not shown). Altogether, these results confirm the functionality in P. tricornutum of the native signal peptide of GLuc from G. princeps. Furthermore, it also demonstrates the functionality of the luciferase in term of enzymatic activity.

g) Immunoblotting Analysis

Wild-type and transformed cells were cultured and the corresponding culture medium were separated from cells and subsequently concentrated by flow filtration.

Aliquotes of wild-type and transformed cells of P. tricornutum culture at exponential phase of growth were collected and cells were separated from the culture medium by centrifugation (10 minutes, 2150 g, 20° C.). The supernatant was filtered using a membrane filter of 0.22 μm pore size and concentrated using a concentration device (MILLIPORE, Microcon, 3 kDa). These samples correspond to the extracellular fraction.

Various volumes (10, 5, 2.5, 1 μL) of extracellular fractions from GLuc transformed cells and 10 μL of extracellular fraction from wild-type were separated by SDS-PAGE using a 12% polyacrylamide gel. The separated proteins were transferred onto nitrocellulose membrane and stained with Ponceau Red in order to control transfer efficiency. The nitrocellulose membrane was blocked overnight in milk 5% dissolved in TBS for immunodetection. Immunodetection was then performed using anti-GLuc (BIOLABS, E8023S) (1:2000 in TBS-T containing milk 1% for 2 h at room temperature). Membranes were then washed with TBS-T (6 times, 5 minutes, room temperature). Binding of anti-GLuc antibody was revealed upon incubation with a secondary horseradish peroxidase-conjugated goat anti-rabbit IgG antibody (SIGMA-ALDRICH, A0545) diluted at 1:10,000 in TBS-T containing milk 1% for 1.5 h at room temperature. Membranes were then washed with TB S-T (6 times, 5 minutes, room temperature) followed by a final wash with TBS (5 minutes, room temperature). Final development of the blots was performed by chemiluminescence method.

As depicted in FIG. 3, no signal was detected in the extracellular fraction from the wild-type cell line. A single band was detected in the extracellular fraction of the full-length GLuc cell line at approximately 18 kDa. It corresponds to the size predicted using a mass prediction software (http://expasy.org/tools/pi_tool.html) after the cleavage of the signal peptide. Indeed, this software predicts a molecular weight at 19.9 kDa for the full-length GLuc and 18.17 kDa for the protein after being cleaved. This result demonstrates the production and the secretion into the culture medium of the recombinant GLuc protein. It also proves the functionality of the native signal peptide from Gaussia princeps when expressed in P. tricornutum.

### Example 2

### Secretion of Enhanced Green Fluorescent Protein (eGFP) in the Culture Medium of Phaeodactylum tricornutum

A second experiment was carried out to test the ability of the exogenous signal peptide from Gaussia princeps luciferase to drive the secretion of the naturally cytosolic eGFP. This chimeric sequence encoded for a 255 amino acids precursor containing a 17 amino acids signal peptide from Gaussia princeps luciferase and a 238 amino acids mature protein.

a) Standard Culture Conditions of Phaeodactylum tricornutum

Phaeodactylum tricornutum strains use in this work were grown and prepared for genetic transformation as in example 1.a).

b) Expression Constructs for the Chimeric eGFP

The vector used for the expression construct of the chimeric eGFP is the same vector used for the expression of luciferase in example 1.b). The chimeric eGFP is encoded by a 768 pb sequence (nucleic acid sequence SEQ ID N°53). Alternatively a 786 pb sequence containing a Histidine tag at the carboxyl-terminus of the protein was also realized (nucleic acid sequence SEQ ID N°54).

The synthesis, digestion and insertion of both sequences in vectors are prepared as the Luciferase sequence in example 1.b). A vector lacking the chimeric eGFP coding sequence is used as control.

c) Genetic Transformation

The genetic transformation carried out in this experiment is described in the previous example 1.c).

d) Immunoblotting Analysis

Aliquotes of wild-type and transformed cells of P. tricornutum culture at exponential phase of growth are collected and cells were separated from the culture medium by centrifugation (10 minutes, 2150 g, 20° C.). Supernatants were filtered using a membrane filter of 0.22 μm pore size and concentrated using a concentration device (MILLIPORE, Microcon, 3 kDa). These samples correspond to the extracellular fraction.

Ten μL of extracellular fractions from eGFP transformed cells and 10 μL of extracellular fraction from wild-type were separated by SDS-PAGE using a 12% polyacrylamide gel. The separated proteins were transferred onto nitrocellulose membrane and stained with Ponceau Red in order to control transfer efficiency. The nitrocellulose membrane was blocked overnight in milk 5% dissolved in TBS for immunodetection. The immunodetection of the chimeric eGFP was performed on the extracellular fractions in the same condition as in example 1.e) except that a horseradish peroxidase-conjugated anti-GFP (Santa Cruz, sc-9996) antibody was used (1:2000 in TBS-T containing milk 1% for 2 h at room temperature).

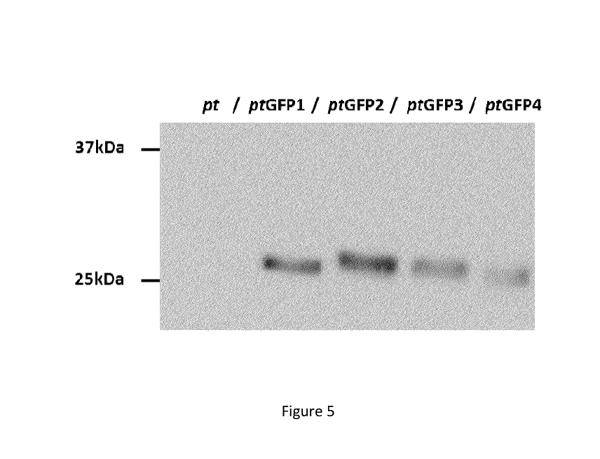

As depicted in FIG. 5, no signal was detected in the extracellular fraction from the wild-type cell line (Pt). A single band was detected in the extracellular fraction of the various clones expressing the chimeric eGFP at approximately 26 kDa (PtGFP1 to PtGFP4). It corresponds to the size predicted using a mass prediction software (http://expasy.org/tools/pi_tool.html) after the cleavage of the signal peptide. Indeed, this software predicts a molecular weight at 28.5 kDa for the full-length chimeric eGFP and 26.8 kDa for the protein after being cleaved. This result demonstrates the production and the secretion into the culture medium of the normally cytosolic eGFP protein when fused to a heterologous peptide signal.

e) Purification of the Secreted Chimeric eGFP

The secreted chimeric eGFP fused to the histidine tag is purified by chromatography method. Culture medium of P. tricornutum at exponential phase of growth is collected and cells are separated from the culture medium by centrifugation (10 minutes, 2150 g, 20° C.). The supernatant is filtered using a membrane filter of 0.22 μm pore size, concentrated 10 times, and buffer-exchanged with 20 mM Tris, pH 9 containing 5 mM imidazole using a concentration device (MILLIPORE, Amicon Ultra-15, 3 kDa). Purification is performed using the AKTA FPLC system (GE Healthcare) and a Ni Sepharose column (GE Healthcare). The column is equilibrated with 20 mM Tris, pH 9.0 buffer containing 5 mM imidazole and the sample is then loaded. The column is washed with buffer containing 10 mM imidazole followed by elution with buffer containing 200 mM imidazole. The peak is collected and loaded on a Sephadex G-50 column equilibrated with 5 mM sodium phosphate buffer, pH 7.4. The desalted protein is collected and concentrated using a concentration device (MILLIPORE, Amicon Ultra-15, 3 kDa).

f) Analysis of the Chimeric eGFP Protein Sequence

Fifteen μL of the purified chimeric eGFP is separated by SDS-PAGE using a 12% polyacrylamide gel. Protein bands are stained with Coomassie brilliant blue CBB R-350 (Amersham Bioscience). The CBB-stained proteins on SDS-PAGE corresponding to chimeric eGFP is excised and digested with sequencing grade modified trypsin (Promega) or arginine-C (Princeton Separations). The gel piece is washed with 50% acetonitrile/0.1 M ammonium bicarbonate, and then dehydrated with acetonitrile. The protein in gel pieces is reduced with 10 mM dithiothreitol and alkylated with 55 mM iodoacetamide. The gel piece is washed once with 20 mM ammonium bicarbonate and dehydrated with acetonitrile. The trypsin solution is added to the gel piece, and the enzyme reaction is allowed to proceed overnight at 37° C. Alternatively, the arginine-C solution is added to the gel piece, and the enzyme reaction is allowed to proceed overnight at room temperature. Both supernatants from trypsin or arginine-C are acidified by adding trifluoroacetic acid and immediately subjected to mass spectrometry or stored in a freezer until analysis. Nano-LC/MS/MS experiments are performed on Q-TOF 2 and Ultima API hybrid mass spectrometers (Waters) equipped with a nano-electrospray ion source and a CapLC system (Waters). The mass spectrometers are operated in data-directed acquisition mode. For protein identification, all MS/MS spectra are searched using the SwissProt data-base.

### Example 3

### Secretion of Murine Erythropoietin in the Culture Medium of Transformed Phaeodactylum tricornutum

A second experiment was carried out in P. tricornutum to test the functionality of exogenous signal peptide. Phaeodactylum tricornutum was transformed with a plasmid containing the murine erythropoietin coding sequence. This sequence encodes for a 192 amino acid precursor that contain a 26 amino acid signal peptide and a 166 amino acid mature protein containing 3 potential N-glycosylation sites.

a) Standard Culture Conditions of Phaeodactylum tricornutum

Phaeodactylum tricornutum strains used in this work were grown and prepared for genetic transformation as in example 1.a).

b) Expression Constructs for EPO

The vector used for the expression construct of murine erythropoietin (EPOm) was the same vector used for the expression of luciferase in example 1.b). Murine erythropoietin is encoded by a 579 pb sequence (SEQ ID N°48).

The synthesis, digestion and insertion of EPOm sequence in the vector were prepared as the Luciferase sequence in example 1.b) Similarly, a vector bearing the EPOm coding sequence lacking the signal peptide was also realized (SEQ ID N°49).

c) Genetic Transformation

The genetic transformation carried out in this experiment is described in the previous example 1.c).

d) Microalgae DNA Extraction

DNA extraction carried out in this experiment is described in the previous example 1.d.

e) Polymerase Chain Reaction (PCR) Analysis

The presence of the transgene was assessed by PCR as described in the previous example 1.e. The sequence of primers used for the amplification EPOm transformed cells were 5′-CACGATGGGTTGTGCAGAAGG-3′ (SEQ ID N° 50) and 5′-CGAAGCAGTGAAGTGAGGCTAC-3′ (SEQ ID N° 51).

Results revealed a single band at 255 bp for cells transformed with the constructs carrying the full-length EPOm or EPOm lacking its signal peptide (data not shown). No band was detected in cells transformed with the control vector. This result validates the incorporation of exogenous gene in the genome of Phaeodactylum tricornutum.

f) Erythropoietin Quantification

EPOm concentration was determined on the extracellular and intracellular fractions of wild-type and transformed cells of P. tricornutum using the ELISA (Enzyme-linked ImmunoSorbent Assay) method. An aliquote of the P. tricornutum culture at exponential phase of growth was collected and cells were separated from the culture medium by centrifugation (10 minutes, 2150 g, 20° C.). The supernatant was then filtered using a membrane filter of 0.22 μm pore size and corresponds to the extracellular fraction. The cell pellet was resuspended with a volume of fresh culture medium equivalent to the initial volume of the aliquote. The cellular suspension was then sonicated during 30 minutes at 4° C. and centrifuged at 4500 g during 5 minutes at 4° C. Supernatant was finally collected and corresponds to the intracellular fraction of P. tricornutum. EPOm quantification was realized on both fractions (intracellular and extracellular) using the ELISA Quantikine Mouse/Rat EPO Immunoassay Kit (R&D SYSTEMS), according to manufacturer's instructions. The lack of interference of the intracellular fraction with the ELISA detection was verified by the addition of a known quantity of recombinant murine EPO (R&D SYSTEMS) to this fraction.

EPOm was mainly detected in the extracellular fraction (0.52 mg/L) when compared to the intracellular fraction (0.02 mg/L) of cells transformed with full-length EPOm construct. Murine EPO could not be detected in both fractions from wild type cells transformed with EPOm construct lacking its signal peptide or wild-type cells. These results revealed that murine EPO was produced with most of the protein being secreted in the culture medium of transformed P. tricornutum. It demonstrates the functionality of a murine signal peptide when expressed in the diatom P. tricornutum.

g) Immunoblotting Analysis

Aliquotes of wild-type and transformed cells of P. tricornutum culture at exponential phase of growth were collected and cells were separated from the culture medium by centrifugation (10 minutes, 2150 g, 20° C.). The supernatant was filtered using a membrane filter of 0.22 μm pore size and concentrated using a concentration device (MILLIPORE, Microcon, 3 kDa). These samples correspond to the extracellular fraction.

The immunodetection of EPO was performed on the extracellular fractions as in example 1.g) by using anti-EPO (R&D SYSTEMS, AF959) antibodies. Binding of said anti-EPO antibody was revealed upon incubation with a secondary horseradish peroxidase-conjugated rabbit anti-goat IgG (SIGMA-ALDRICH, A8919) in the same condition as in example 1.e).

As depicted in FIG. 4, no band was visible in the sample from the wild-type cell line. A single band was detected in the extracellular fraction purified from the transformed cells with a molecular weight around 25 kDa. As expected, a band at 34 kDa was detected for the commercial recombinant murine EPO used as control. Erythropoietin possesses 3 potential N-glycosylation sites. Since the predicted molecular weight of the amino acids backbone of EPO is 20 kDa, this result suggested that the protein was glycosylated. The difference of molecular weight between native murine EPO and EPO produced in P. tricornutum could originate from a difference in the glycan moieties. This result also strongly suggested that EPO followed the classical ER-golgi secretory pathway allowing the glycosylation of this protein.

Altogether, data from the ELISA and western blot experiments prove that EPO was produced and secreted in the culture medium of P. tricornutum. These results also demonstrate the functionality of the native signal peptide of the murine EPO.

### Example 4

### Secretion of Human Interleukin-2 in the Culture Medium of Transformed Phaeodactylum tricornutum

A third experiment is carried out in P. tricornutum to test the functionality of exogenous signal peptides. Phaeodactylum tricornutum is transformed with a plasmid containing the human interleukin-2 coding sequence. This sequence encodes for a 153 amino acid precursor that contain a 20 amino acid signal peptide and a 133 amino acid mature protein containing one potential O-glycosylation site.

a) Standard Culture Conditions of Phaeodactylum tricornutum

Phaeodactylum tricornutum strains use in this work were grown and prepared for genetic transformation as in example 1.a).

b) Expression Constructs for IL-2

The vector used for the expression construct of human IL-2 (IL-2) is the same vector used for the expression of luciferase in example 1.b). Human interleukin-2 is encoded by a 462 pb sequence (SEQ ID N°4).

The synthesis, digestion and insertion of human IL-2 sequences in vectors are prepared as the Luciferase sequence in example 1.b). Similarly, a vector bearing the IL-2 coding sequence lacking the signal peptide is also realized (SEQ ID N°52). A vector lacking the IL-2 coding sequence is used as control.

c) Genetic Transformation

The genetic transformation carried out in this experiment is described in the previous example 1.c).

d) Interleukin-2 Quantification

IL-2 concentrations are determined on the extracellular and intracellular fractions of wild-type and P. tricornutum transformed by full-length IL-2 or IL-2 lacking its signal peptide. An aliquote of the P. tricornutum culture at exponential phase of growth is collected and processed to collect both extracellular and intracellular fractions as described in example 2.f). IL-2 quantification is realized using the ELISA Quantikine Human IL-2 Immunoassay Kit (R&D SYSTEMS), according to manufacturer's instructions.

e) Immunoblotting of the Secreted IL-2

Aliquotes of wild-type and transformed cells of P. tricornutum culture at exponential phase of growth are collected and cells are separated from the culture medium by centrifugation (10 minutes, 2150 g, 20° C.). Supernatants are filtered using a membrane filter of 0.22 μm pore size and concentrated using a concentration device (MILLIPORE, Microcon, 3 kDa). These samples correspond to the extracellular fraction.

The immunodetection of IL-2 is performed on various volume of purified fractions (5, 10, 15 μL) by using anti-IL-2 (R&D SYSTEMS, AB-202-NA) antibodies. Binding of said anti-IL-2 antibody is revealed upon incubation with a secondary horseradish peroxidase-conjugated rabbit anti-goat IgG (SIGMA-ALDRICH, A8919) in the same condition as in example 1.e).

f) Purification of the Secreted IL-2

The secreted IL-2 is purified by chromatography method. Culture medium of P. tricornutum at exponential phase of growth is collected and cells are separated from the culture medium by centrifugation (10 minutes, 2150 g, 20° C.). The supernatant is filtered using a membrane filter of 0.22 μm pore size, concentrated 10 times, and buffer-exchanged with 25 mM ammonium acetate, pH 5 using a concentration device (MILLIPORE, Amicon Ultra-15, 3 kDa).

Purification is performed using the AKTA FPLC system (GE Healthcare) and a CM Sepharose column (GE Healthcare). The column is equilibrated with 25 mM ammonium acetate, pH 5. The sample is then loaded to the column The column is washed extensively, and bound IL-2 is eluted with a step gradient of 0-1 M sodium chloride in 25 mM ammonium acetate, pH 5. The peak is collected and loaded on a Sephadex G-50 column equilibrated with 5 mM sodium phosphate buffer, pH 7.4. The desalted protein is collected and concentrated using a concentration device (MILLIPORE, Amicon Ultra-15, 3 kDa). Concentration of IL-2 in collected fractions is determined by ELISA method and the purity of IL-2 is assessed by immunoblotting analysis.

g) Analysis of IL-2 Protein Sequence

Fifteen μL of IL-2 purified from the extracellular medium is separated by SDS-PAGE using a 12% polyacrylamide gel. Protein bands are stained with Coomassie brilliant blue CBB R-350 (Amersham Bioscience). The CBB-stained proteins on SDS-PAGE corresponding to IL-2 is excised and digested with sequencing grade modified trypsin (Promega). The gel piece is washed with 50% acetonitrile/0.1 M ammonium bicarbonate, and then dehydrated with acetonitrile. The protein in gel pieces is reduced with 10 mM dithiothreitol and alkylated with 55 mM iodoacetamide. The gel piece is washed once with 20 mM ammonium bicarbonate and dehydrated with acetonitrile. The trypsin solution is added to the gel piece, and the enzyme reaction is allowed to proceed overnight at 37° C. After digestion, the supernatant is acidified by adding trifluoroacetic acid and immediately subjected to mass spectrometry or stored in a freezer until analysis. Nano-LC/MS/MS experiments are performed on Q-TOF 2 and Ultima API hybrid mass spectrometers (Waters) equipped with a nano-electrospray ion source and a CapLC system (Waters). The mass spectrometers are operated in data-directed acquisition mode. For protein identification, all MS/MS spectra are searched using the SwissProt data-base.

### Example 5

### Expression of the O-Glucocerebrosidase also Called β-Glucosidase Acid (GBA) Protein

a) Standard Culture Conditions of Phaeodactylum tricornutum

Diatoms are grown and prepared for the genetic transformation as in example 1.a). The conditions of culture may be adapted to the species used for the secretion of PROTEIN.

b) Expression Constructs for the Protein of Therapeutic Interest

The vector used for the expression construct of GBA is the same vector used in example 1.b). GBA is encoded by the nucleic acid sequence SEQ ID N°29 as listed in Table I. The synthesis, digestion and insertion of GBA sequence in the vector are prepared as in example 1.b).

c) Genetic Transformation

The transformation carried out on diatoms is described in the example 1.c).

d) Protein Quantification

GBA concentration is determined on the extracellular and intracellular fractions of transformed diatoms by using the ELISA method as described in example 2.0.

e) Immunoblotting Analysis

The immunodetection of GBA is performed as in example 1.g) by using anti-GBA antibodies. Binding of said anti-GBA antibodies is revealed upon incubation with a secondary antibody directed against anti-GBA antibodies.

### Example 6

### Expression of Proteins of Therapeutic Interest as Listed in Table I

The term “PROTEIN” corresponds herein to the name of the protein of therapeutic interest to be secreted in the extracellular medium of diatoms, said name being listed in Table I, and derivatives thereof.

f) Standard Culture Conditions of Phaeodactylum tricornutum

Diatoms are grown and prepared for the genetic transformation as in example 1.a). The conditions of culture may be adapted to the species used for the secretion of PROTEIN.

g) Expression Constructs for the Protein of Therapeutic Interest

The vector used for the expression construct of PROTEIN is the same vector used in example 1.b). PROTEIN is encoded by the nucleic acid sequence listed in Table I. The synthesis, digestion and insertion of PROTEIN sequence in the vector are prepared as in example 1.b).

h) Genetic Transformation

The transformation carried out on diatoms is described in the example 1.c).

i) Protein Quantification

PROTEIN concentration is determined on the extracellular and intracellular fractions of transformed diatoms by using the ELISA method as described in example 2.0.

j) Immunoblotting Analysis

The immunodetection of PROTEIN is performed as in example 1.g) by using anti-PROTEIN antibodies. Binding of said anti-PROTEIN antibodies is revealed upon incubation with a secondary antibody directed against anti-PROTEIN antibodies.

1. A transformed diatom comprising a nucleic acid sequence operatively linked to a promoter, wherein said nucleic acid sequence encodes an amino acid sequence comprising:
(i) an heterologous signal peptide; and
(ii) a polypeptide,
said heterologous signal peptide leading to the secretion of said polypeptide in the extracellular medium of said transformed diatom.

2. The transformed diatom according to claim 1, wherein said diatom is selected from the group comprising Phaeodactylacaeae diatoms.

3. The transformed diatom according to claim 1, wherein said diatom is Phaeodactylum tricornutum.

4. The transformed diatom according to claim 1, wherein
said polypeptide is a heterologous polypeptide, and
said heretologous signal peptide is the signal peptide of said heterologous polypeptide, said signal peptide leading to the secretion of said polypeptide in the extracellular medium of the organism of said polypeptide.

5. The transformed diatom according to claim 1, wherein the polypeptide is an animal polypeptide of animal origin, preferably a mammalian polypeptide of mammalian origin and most preferably a human polypeptide of human origin.

6. The transformed diatom according to claim 1, wherein said polypeptide is selected from the group comprising erythropoietin, cytokines such as interferons, antibodies and their fragments, coagulation factors, hormones, beta-glucocerebrosidase, pentraxin-3, anti-TNFs, α-glucosidase acide, α-L-iduronidase and derivatives thereof.

7. The transformed diatom according to claim 1, wherein said nucleic acid sequence is selected from the group comprising the nucleic acid sequences as listed in Table I and derivatives thereof.

8. A method for producing a polypeptide which is secreted in the extracellular medium, said method comprising the steps of:
(i) culturing a transformed diatom as defined in claim 1;
(ii) harvesting the extracellular medium of said culture; and
(iii) purifying the secreted polypeptide in said extracellular medium.

9. The method according to claim 8, wherein said method comprises a step (iv) of determining the glycosylation pattern of said polypeptide.

10. The method according to claim 8, wherein said method leads to the secretion in the extracellular medium of at least 25%, 50%, 75% or 90% of the polypeptide expressed in said diatom.

11. The method according to claim 9, wherein said method leads to the secretion in the extracellular medium of at least 25%, 50%, 75% or 90% of the polypeptide expressed in said diatom.

12. The transformed diatom according to claim 2, wherein said diatom is Phaeodactylum tricornutum.

13. The transformed diatom according to claim 2, wherein
said polypeptide is a heterologous polypeptide, and
said heterologous signal peptide is the signal peptide of said heterologous polypeptide, said signal peptide leading to the secretion of said polypeptide in the extracellular medium of the organism of said polypeptide.

14. The transformed diatom according to claim 3, wherein
said polypeptide is a heterologous polypeptide, and
said heterologous signal peptide is the signal peptide of said heterologous polypeptide, said signal peptide leading to the secretion of said polypeptide in the extracellular medium of the organism of said polypeptide.


SEQUENCE LISTING (54 sequences)

SEQ ID NO: 1 - DNA, 564 bp, Homo sapiens
atgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactga

SEQ ID NO: 2 - DNA, 567 bp, Homo sapiens
atggccttgacctttgctttactggtggccctcctggtgctcagctgcaagtcaagctgctctgtgggctgtgatctgcctcaaacccacagcctgggtagcaggaggaccttgatgctcctggcacagatgaggagaatctctcttttctcctgcttgaaggacagacatgactttggatttccccaggaggagtttggcaaccagttccaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaatga

SEQ ID NO: 3 - DNA, 600 bp, Homo sapiens
atgaactgtgtttgccgcctggtcctggtcgtgctgagcctgtggccagatacagctgtcgcccctgggccaccacctggcccccctcgagtttccccagaccctcgggccgagctggacagcaccgtgctcctgacccgctctctcctggcggacacgcggcagctggctgcacagctgagggacaaattcccagctgacggggaccacaacctggattccctgcccaccctggccatgagtgcgggggcactgggagctctacagctcccaggtgtgctgacaaggctgcgagcggacctactgtcctacctgcggcacgtgcagtggctgcgccgggcaggtggctcttccctgaagaccctggagcccgagctgggcaccctgcaggcccgactggaccggctgctgcgccggctgcagctcctgatgtcccgcctggccctgccccagccacccccggacccgccggcgcccccgctggcgcccccctcctcagcctgggggggcatcagggccgcccacgccatcctgggggggctgcacctgacacttgactgggccgtgaggggactgctgctgctgaagactcggctgtga

SEQ ID NO: 4 - DNA, 462 bp, Homo sapiens
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 5 - DNA, 639 bp, Homo sapiens
atgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtag

SEQ ID NO: 6 - DNA, 489 bp, Homo sapiens
atgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattcctga

SEQ ID NO: 7 - DNA, 333 bp, Homo sapiens
atggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaactag

SEQ ID NO: 8 - DNA, 540 bp, Homo sapiens
atgaaaagcatttactttgtggctggattatttgtaatgctggtacaaggcagctggcaacgttcccttcaagacacagaggagaaatccagatcattctcagcttcccaggcagacccactcagtgatcctgatcagatgaacgaggacaagcgccattcacagggcacattcaccagtgactacagcaagtatctggactccaggcgtgcccaagattttgtgcagtggttgatgaataccaagaggaacaggaataacattgccaaacgtcacgatgaatttgagagacatgctgaagggacctttaccagtgatgtaagttcttatttggaaggccaagctgccaaggaattcattgcttggctggtgaaaggccgaggaaggcgagatttcccagaagaggtcgccattgttgaagaacttggccgcagacatgctgatggttctttctctgatgagatgaacaccattcttgataatcttgccgccagggactttataaactggttgattcagaccaaaatcactgacaggtga

SEQ ID NO: 9 - DNA, 582 bp, Homo sapiens
atgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagatga

SEQ ID NO: 10 - DNA, 654 bp, Homo sapiens
atggctacaggctcccggacgtccctgctcctggcttttggcctgctctgcctgccctggcttcaagagggcagtgccttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctag

SEQ ID NO: 11 - DNA, 609 bp, Homo sapiens
atggctacaggctcccggacgtccctgctcctggcttttggcctgctctgcctgccctggcttcaagagggcagtgccttcccaaccattcccttatccaggctttttgacaacgctatgctccgcgcccatcgtctgcaccagctggcctttgacacctaccaggagtttaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctag

SEQ ID NO: 12 - DNA, 534 bp, Homo sapiens
atggctacaggctcccggacgtccctgctcctggcttttggcctgctctgcctgccctggcttcaagagggcagtgccttcccaaccattcccttatccaggctttttgacaacgctatgctccgcgcccatcgtctgcaccagctggcctttgacacctaccaggagtttaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctag

SEQ ID NO: 13 - DNA, 369 bp, Homo sapiens
atggctacaggctcccggacgtccctgctcctggcttttggcctgctctgcctgccctggcttcaagagggcagtgccttcccaaccattcccttatccaggctttttgacaacgctatgctccgcgcccatcgtctgcaccagctggcctttgacacctaccaggagtttaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctag

SEQ ID NO: 14 - DNA, 93 bp, Homo sapiens
atggctacagaggctggaagatggcagcccccggactgggcagatcttcaagcagacctacagcaagttcgacacaaactcacacaacgatga

SEQ ID NO: 15 - DNA, 435 bp, Homo sapiens
atgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtga

SEQ ID NO: 16 - DNA, 624 bp, Homo sapiens
atggctggacctgccacccagagccccatgaagctgatggccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctggtgagtgagtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagccctga

SEQ ID NO: 17 - DNA, 351 bp, Homo sapiens
atggattactacagaaaatatgcagctatctttctggtcacattgtcggtgtttctgcatgttctccattccgctcctgatgtgcaggattgcccagaatgcacgctacaggaaaacccattcttctcccagccgggtgccccaatacttcagtgcatgggctgctgcttctctagagcatatcccactccactaaggtccaagaagacgatgttggtccaaaagaacgtcacctcagagtccacttgctgtgtagctaaatcatataacagggtcacagtaatggggggtttcaaagtggagaaccacacggcgtgccactgcagtacttgttattatcacaaatcttaa

SEQ ID NO: 18 - DNA, 390 bp, Homo sapiens
atgaagacactccagtttttcttccttttctgttgctggaaagcaatctgctgcaatagctgtgagctgaccaacatcaccattgcaatagagaaagaagaatgtcgtttctgcataagcatcaacaccacttggtgtgctggctactgctacaccagggatctggtgtataaggacccagccaggcccaaaatccagaaaacatgtaccttcaaggaactggtatacgaaacagtgagagtgcccggctgtgctcaccatgcagattccttgtatacatacccagtggccacccagtgtcactgtggcaagtgtgacagcgacagcactgattgtactgtgcgaggcctggggcccagctactgctcctttggtgaaatgaaagaataa

SEQ ID NO: 19 - DNA, 498 bp, Homo sapiens
atggagatgttccaggggctgctgctgttgctgctgctgagcatgggcgggacatgggcatccaaggagccgcttcggccacggtgccgccccatcaatgccaccctggctgtggagaaggagggctgccccgtgtgcatcaccgtcaacaccaccatctgtgccggctactgccccaccatgacccgcgtgctgcagggggtcctgccggccctgcctcaggtggtgtgcaactaccgcgatgtgcgcttcgagtccatccggctccctggctgcccgcgcggcgtgaaccccgtggtctcctacgccgtggctctcagctgtcaatgtgcactctgccgccgcagcaccactgactgcgggggtcccaaggaccaccccttgacctgtgatgacccccgcttccaggactcctcttcctcaaaggcccctccccccagccttccaagcccatcccgactcccggggccctcggacaccccgatcctcccacaataa

SEQ ID NO: 20 - DNA, 417 bp, Homo sapiens
atgactgctctctttctgatgtccatgctttttggccttacatgtgggcaagcgatgtctttttgtattccaactgagtatacaatgcacatcgaaaggagagagtgtgcttattgcctaaccatcaacaccaccatctgtgctggatattgtatgacacgggatatcaatggcaaactgtttcttcccaaatatgctctgtcccaggatgtttgcacatatagagacttcatctacaggactgtagaaataccaggatgcccactccatgttgctccctatttttcctatcctgttgctttaagctgtaagtgtggcaagtgcaatactgactatagtgactgcatacatgaagccatcaagacaaactactgtaccaaacctcagaagtcttatctggtaggattttctgtctaa

SEQ ID NO: 21 - DNA, 426 bp, Homo sapiens
atggagatgctccaggggctgctgctgttgctgctgctgagcatgggcggggcatgggcatccagggagccgcttcggccatggtgccaccccatcaatgccatcctggctgtcgagaaggagggctgcccagtgtgcatcaccgtcaacaccaccatctgtgccggctactgccccaccatgatgcgcgtgctgcaggcggtcctgccgcccctgcctcaggtggtgtgcacctaccgtgatgtgcgcttcgagtccatccggctccctggctgcccgcgtggtgtggaccccgtggtctccttccctgtggctctcagctgtcgctgtggaccctgccgccgcagcacctctgactgtgggggtcccaaagaccaccccttgacctgtgaccacccccaactctcaggcctcctcttcctctaa

SEQ ID NO: 22 - DNA, 1869 bp, Homo sapiens
atggcgcacgtccgaggcttgcagctgcctggctgcctggccctggctgccctgtgtagccttgtgcacagccagcatgtgttcctggctcctcagcaagcacggtcgctgctccagcgggtccggcgagccaacaccttcttggaggaggtgcgcaagggcaacctggagcgagagtgcgtggaggagacgtgcagctacgaggaggccttcgaggctctggagtcctccacggctacggatgtgttctgggccaagtacacagcttgtgagacagcgaggacgcctcgagataagcttgctgcatgtctggaaggtaactgtgctgagggtctgggtacgaactaccgagggcatgtgaacatcacccggtcaggcattgagtgccagctatggaggagtcgctacccacataagcctgaaatcaactccactacccatcctggggccgacctacaggagaatttctgccgcaaccccgacagcagcaccacgggaccctggtgctacactacagaccccaccgtgaggaggcaggaatgcagcatccctgtctgtggccaggatcaagtcactgtagcgatgactccacgctccgaaggctccagtgtgaatctgtcacctccattggagcagtgtgtccctgatcgggggcagcagtaccaggggcgcctggcggtgaccacacatgggctcccctgcctggcctgggccagcgcacaggccaaggccctgagcaagcaccaggacttcaactcagctgtgcagctggtggagaacttctgccgcaacccagacggggatgaggagggcgtgtggtgctatgtggccgggaagcctggcgactttgggtactgcgacctcaactattgtgaggaggccgtggaggaggagacaggagatgggctggatgaggactcagacagggccatcgaagggcgtaccgccaccagtgagtaccagactttcttcaatccgaggacctttggctcgggagaggcagactgtgggctgcgacctctgttcgagaagaagtcgctggaggacaaaaccgaaagagagctcctggaatcctacatcgacgggcgcattgtggagggctcggatgcagagatcggcatgtcaccttggcaggtgatgcttttccggaagagtccccaggagctgctgtgtggggccagcctcatcagtgaccgctgggtcctcaccgccgcccactgcctcctgtacccgccctgggacaagaacttcaccgagaatgaccttctggtgcgcattggcaagcactcccgcaccaggtacgagcgaaacattgaaaagatatccatgttggaaaagatctacatccaccccaggtacaactggcgggagaacctggaccgggacattgccctgatgaagctgaagaagcctgttgccttcagtgactacattcaccctgtgtgtctgcccgacagggagacggcagccagcttgctccaggctggatacaaggggcgggtgacaggctggggcaacctgaaggagacgtggacagccaacgttggtaaggggcagcccagtgtcctgcaggtggtgaacctgcccattgtggagcggccggtctgcaaggactccacccggatccgcatcactgacaacatgttctgtgctggttacaagcctgatgaagggaaacgaggggatgcctgtgaaggtgacagtgggggaccctttgtcatgaagagcccctttaacaaccgctggtatcaaatgggcatcgtctcatggggtgaaggctgtgaccgggatgggaaatatggcttctacacacatgtgttccgcctgaagaagtggatacagaaggtcattgatcagtttggagagtag

SEQ ID NO: 23 - DNA, 1401 bp, Homo sapiens
atggtctcccaggccctcaggctcctctgccttctgcttgggcttcagggctgcctggctgcaggcggggtcgctaaggcctcaggaggagaaacacgggacatgccgtggaagccggggcctcacagagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggccgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttccctag

SEQ ID NO: 24 - DNA, 7056 bp, Homo sapiens
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggttcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgagggggcatatgctccagtacttcaagattttaggtcattaaatgattcaacaaatagaacaaagaaacacacagctcatttctcaaaaaaaggggaggaagaaaacttggaaggcttgggaaatcaaaccaagcaaattgtagagaaatatgcatgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 25 - DNA, 1389 bp, Homo sapiens
atgcagcgcgtgaacatgatcatggcagaatcaccaagcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaaccacgagaagtttttgaaaacactgaaaagacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgaggctgtttttcctgatgtggactatgtaaatcctactgaagctgaaaccattttggataacatcactcaaggcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgagcaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctgggcaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaa

SEQ ID NO: 26 - DNA, 1551 bp, Homo sapiens
atggatgcaatgaagagagggctctgctgtgtgctgctgctgtgtggagcagtcttcgtttcgcccagccaggaaatccatgcccgattcagaagaggagccagatcttaccaaggttgcagcgagccaaggtgtttcaacgggggcacctgccagcaggccctgtacttctcagatttcgtgtgccagtgccccgaaggatttgctgggaagtgctgtgaaatagataccagggccacgtgctacgaggaccagggcatcagctacaggggcacgtggagcacagcggagagtggcgccgagtgcaccaactggaacagcagcgcgttggcccagaagccctacagcgggcggaggccagacgccatcaggctgggcctggggaaccacaactactgcagaaacccagatcgagactcaaagccctggtgctacgtctttaaggcggggaagtacagctcagagttctgcagcacccctgcctgctctgagggaaacagtgactgctactttgggaatgggtcagcctaccgtggcacgcacagcctcaccgagtcgggtgcctcctgcctcccgtggaattccatgatcctgataggcaaggtttacacagcacagaaccccagtgcccaggcactgggcctgggcaaacataattactgccggaatcctgatggggatgccaagccctggtgccacgtgctgaagaaccgcaggctgacgtgggagtactgtgatgtgccctcctgctccacctgcggcctgagacagtacagccagcctcagtttcgcatcaaaggagggctcttcgccgacatcgcctcccacccctggcaggctgccatctttgccaagcacaggaggtcgcccggagagcggttcctgtgcgggggcatactcatcagctcctgctggattctctctgccgcccactgcttccaggagaggtttccgccccaccacctgacggtgatcttgggcagaacataccgggtggtccctggcgaggaggagcagaaatttgaagtcgaaaaatacattgtccataaggaattcgatgatgacacttacgacaatgacattgcgctgctgcagctgaaatcggattcgtcccgctgtgcccaggagagcagcgtggtccgcactgtgtgccttcccccggcggacctgcagctgccggactggacggagtgtgagctctccggctacggcaagcatgaggccttgtctcctttctattcggagcggctgaaggaggctcatgtcagactgtacccatccagccgctgcacatcacaacatttacttaacagaacagtcaccgacaacatgctgtgtgctggagacactcggagcggcgggccccaggcaaacttgcacgacgcctgccagggcgattcgggaggccccctggtgtgtctgaacgatggccgcatgactttggtgggcatcatcagctggggcctgggctgtggacagaaggatgtcccgggtgtgtacaccaaggttaccaactacctagactggattcgtgacaacatgcgaccgtga

SEQ ID NO: 27 - DNA, 1689 bp, Homo sapiens
atggatgcaatgaagagagggctctgctgtgtgctgctgctgtgtggagcagtcttcgtttcgcccagccaggaaatccatgcccgattcagaagaggagccagatcttaccaagtgatctgcagagatgaaaaaacgcagatgatataccagcaacatcagtcatggctgcgccctgtgctcagaagcaaccgggtggaatattgctggtgcaacagtggcagggcacagtgccactcagtgcctgtcaaaagttgcagcgagccaaggtgtttcaacgggggcacctgccagcaggccctgtacttctcagatttcgtgtgccagtgccccgaaggatttgctgggaagtgctgtgaaatagataccagggccacgtgctacgaggaccagggcatcagctacaggggcacgtggagcacagcggagagtggcgccgagtgcaccaactggaacagcagcgcgttggcccagaagccctacagcgggcggaggccagacgccatcaggctgggcctggggaaccacaactactgcagaaacccagatcgagactcaaagccctggtgctacgtctttaaggcggggaagtacagctcagagttctgcagcacccctgcctgctctgagggaaacagtgactgctactttgggaatgggtcagcctaccgtggcacgcacagcctcaccgagtcgggtgcctcctgcctcccgtggaattccatgatcctgataggcaaggtttacacagcacagaaccccagtgcccaggcactgggcctgggcaaacataattactgccggaatcctgatggggatgccaagccctggtgccacgtgctgaagaaccgcaggctgacgtgggagtactgtgatgtgccctcctgctccacctgcggcctgagacagtacagccagcctcagtttcgcatcaaaggagggctcttcgccgacatcgcctcccacccctggcaggctgccatctttgccaagcacaggaggtcgcccggagagcggttcctgtgcgggggcatactcatcagctcctgctggattctctctgccgcccactgcttccaggagaggtttccgccccaccacctgacggtgatcttgggcagaacataccgggtggtccctggcgaggaggagcagaaatttgaagtcgaaaaatacattgtccataaggaattcgatgatgacacttacgacaatgacattgcgctgctgcagctgaaatcggattcgtcccgctgtgcccaggagagcagcgtggtccgcactgtgtgccttcccccggcggacctgcagctgccggactggacggagtgtgagctctccggctacggcaagcatgaggccttgtctcctttctattcggagcggctgaaggaggctcatgtcagactgtacccatccagccgctgcacatcacaacatttacttaacagaacagtcaccgacaacatgctgtgtgctggagacactcggagcggcgggccccaggcaaacttgcacgacgcctgccagggcgattcgggaggccccctggtgtgtctgaacgatggccgcatgactttggtgggcatcatcagctggggcctgggctgtggacagaaggatgtcccgggtgtgtacaccaaggttaccaactacctagactggattcgtgacaacatgcgaccgtga

SEQ ID NO: 28 - DNA, 1386 bp, Homo sapiens
atgtggcagctcacaagcctcctgctgttcgtggccacctggggaatttccggcacaccagctcctcttgactcagtgttctccagcagcgagcgtgcccaccaggtgctgcggatccgcaaacgtgccaactccttcctggaggagctccgtcacagcagcctggagcgggagtgcatagaggagatctgtgacttcgaggaggccaaggaaattttccaaaatgtggatgacacactggccttctggtccaagcacgtcgacggtgaccagtgcttggtcttgcccttggagcacccgtgcgccagcctgtgctgcgggcacggcacgtgcatcgacggcatcggcagcttcagctgcgactgccgcagcggctgggagggccgcttctgccagcgcgaggtgagcttcctcaattgctcgctggacaacggcggctgcacgcattactgcctagaggaggtgggctggcggcgctgtagctgtgcgcctggctacaagctgggggacgacctcctgcagtgtcaccccgcagtgaagttcccttgtgggaggccctggaagcggatggagaagaagcgcagtcacctgaaacgagacacagaagaccaagaagaccaagtagatccgcggctcattgatgggaagatgaccaggcggggagacagcccctggcaggtggtcctgctggactcaaagaagaagctggcctgcggggcagtgctcatccacccctcctgggtgctgacagcggcccactgcatggatgagtccaagaagctccttgtcaggcttggagagtatgacctgcggcgctgggagaagtgggagctggacctggacatcaaggaggtcttcgtccaccccaactacagcaagagcaccaccgacaatgacatcgcactgctgcacctggcccagcccgccaccctctcgcagaccatagtgcccatctgcctcccggacagcggccttgcagagcgcgagctcaatcaggccggccaggagaccctcgtgacgggctggggctaccacagcagccgagagaaggaggccaagagaaaccgcaccttcgtcctcaacttcatcaagattcccgtggtcccgcacaatgagtgcagcgaggtcatgagcaacatggtgtctgagaacatgctgtgtgcgggcatcctcggggaccggcaggatgcctgcgagggcgacagtggggggcccatggtcgcctccttccacggcacctggttcctggtgggcctggtgagctggggtgagggctgtgggctccttcacaactacggcgtttacaccaaagtcagccgctacctcgactggatccatgggcacatcagagacaaggaagccccccagaagagctgggcaccttag

SEQ ID NO: 29 - DNA, 1611 bp, Homo sapiens
atggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtga

SEQ ID NO: 30 - DNA, 1290 bp, Homo sapiens
atgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 31 - DNA, 2859 bp, Homo sapiens
atgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 32 - DNA, 1296 bp, Homo sapiens
atgcacgtgcgctcactgcgagctgcggcgccgcacagcttcgtggcgctctgggcacccctgttcctgctgcgctccgccctggccgacttcagcctggacaacgaggtgcactcgagcttcatccaccggcgcctccgcagccaggagcggcgggagatgcagcgcgagatcctctccattttgggcttgccccaccgcccgcgcccgcacctccagggcaagcacaactcggcacccatgttcatgctggacctgtacaacgccatggcggtggaggagggcggcgggcccggcggccagggcttctcctacccctacaaggccgtcttcagtacccagggcccccctctggccagcctgcaagatagccatttcctcaccgacgccgacatggtcatgagcttcgtcaacctcgtggaacatgacaaggaattcttccacccacgctaccaccatcgagagttccggtttgatctttccaagatcccagaaggggaagctgtcacggcagccgaattccggatctacaaggactacatccgggaacgcttcgacaatgagacgttccggatcagcgtttatcaggtgctccaggagcacttgggcagggaatcggatctcttcctgctcgacagccgtaccctctgggcctcggaggagggctggctggtgtttgacatcacagccaccagcaaccactgggtggtcaatccgcggcacaacctgggcctgcagctctcggtggagacgctggatgggcagagcatcaaccccaagttggcgggcctgattgggcggcacgggccccagaacaagcagcccttcatggtggctttcttcaaggccacggaggtccacttccgcagcatccggtccacggggagcaaacagcgcagccagaaccgctccaagacgcccaagaaccaggaagccctgcggatggccaacgtggcagagaacagcagcagcgaccagaggcaggcctgtaagaagcacgagctgtatgtcagcttccgagacctgggctggcaggactggatcatcgcgcctgaaggctacgccgcctactactgtgagggggagtgtgccttccctctgaactcctacatgaacgccaccaaccacgccatcgtgcagacgctggtccacttcatcaacccggaaacggtgcccaagccctgctgtgcgcccacgcagctcaatgccatctccgtcctctacttcgatgacagctccaacgtcatcctgaagaaatacagaaacatggtggtccgggcctgtggctgccactag

SEQ ID NO: 33 - DNA, 1191 bp, Homo sapiens
atggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggaaaagggcatcctctccacaaaagagaaaaacgtcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgctag

SEQ ID NO: 34 - DNA, 1962 bp, Homo sapiens
atgcgtcccctgcgcccccgcgccgcgctgctggcgctcctggcctcgctcctggccgcgcccccggtggccccggccgaggccccgcacctggtgcatgtggacgcggcccgcgcgctgtggcccctgcggcgcttctggaggagcacaggcttctgccccccgctgccacacagccaggctgaccagtacgtcctcagctgggaccagcagctcaacctcgcctatgtgggcgccgtccctcaccgcggcatcaagcaggtccggacccactggctgctggagcttgtcaccaccagggggtccactggacggggcctgagctacaacttcacccacctggacgggtacctggaccttctcagggagaaccagctcctcccagggtttgagctgatgggcagcgcctcgggccacttcactgactttgaggacaagcagcaggtgtttgagtggaaggacttggtctccagcctggccaggagatacatcggtaggtacggactggcgcatgtttccaagtggaacttcgagacgtggaatgagccagaccaccacgactttgacaacgtctccatgaccatgcaaggcttcctgaactactacgatgcctgctcggagggtctgcgcgccgccagccccgccctgcggctgggaggccccggcgactccttccacaccccaccgcgatccccgctgagctggggcctcctgcgccactgccacgacggtaccaacttcttcactggggaggcgggcgtgcggctggactacatctccctccacaggaagggtgcgcgcagctccatctccatcctggagcaggagaaggtcgtcgcgcagcagatccggcagctcttccccaagttcgcggacacccccatttacaacgacgaggcggacccgctggtgggctggtccctgccacagccgtggagggcggacgtgacctacgcggccatggtggtgaaggtcatcgcgcagcatcagaacctgctactggccaacaccacctccgccttcccctacgcgctcctgagcaacgacaatgccttcctgagctaccacccgcaccccttcgcgcagcgcacgctcaccgcgcgcttccaggtcaacaacacccgcccgccgcacgtgcagctgttgcgcaagccggtgctcacggccatggggctgctggcgctgctggatgaggagcagctctgggccgaagtgtcgcaggccgggaccgtcctggacagcaaccacacggtgggcgtcctggccagcgcccaccgcccccagggcccggccgacgcctggcgcgccgcggtgctgatctacgcgagcgacgacacccgcgcccaccccaaccgcagcgtcgcggtgaccctgcggctgcgcggggtgccccccggcccgggcctggtctacgtcacgcgctacctggacaacgggctctgcagccccgacggcgagtggcggcgcctgggccggcccgtcttccccacggcagagcagttccggcgcatgcgcgcggctgaggacccggtggccgcggcgccccgccccttacccgccggcggccgcctgaccctgcgccccgcgctgcggctgccgtcgcttttgctggtgcacgtgtgtgcgcgccccgagaagccgcccgggcaggtcacgcggctccgcgccctgcccctgacccaagggcagctggttctggtctggtcggatgaacacgtgggctccaagtgcctgtggacatacgagatccagttctctcaggacggtaaggcgtacaccccggtcagcaggaagccatcgaccttcaacctctttgtgttcagcccagacacaggtgctgtctctggctcctaccgagttcgagccctggactactgggcccgaccaggccccttctcggaccctgtgccgtacctggaggtccctgtgccaagagggcccccatccccgggcaatccatga

SEQ ID NO: 35 - DNA, 1398 bp, Homo sapiens
atgctgccactttggactctttcactgctgctgggagcagtagcaggaaaagaagtttgctacgaaagactcggctgcttcagtgatgactccccatggtcaggaattacggaaagacccctccatatattgccttggtctccaaaagatgtcaacacccgcttcctcctatatactaatgagaacccaaacaactttcaagaagttgccgcagattcatcaagcatcagtggctccaatttcaaaacaaatagaaaaactcgctttattattcatggattcatagacaagggagaagaaaactggctggccaatgtgtgcaagaatctgttcaaggtggaaagtgtgaactgtatctgtgtggactggaaaggtggctcccgaactggatacacacaagcctcgcagaacatcaggatcgtgggagcagaagtggcatattttgttgaatttcttcagtcggcgttcggttactcaccttccaatgtgcatgtcattggccacagcctgggtgcccacgctgctggggaggctggaaggagaaccaatgggaccattggacgcatcacagggttggacccagcagaaccttgctttcagggcacacctgaattagtccgattggaccccagcgatgccaaatttgtggatgtaattcacacggatggtgcccccatagtccccaatttggggtttggaatgagccaagtcgtgggccacctagatttctttccaaatggaggagtggaaatgcctggatgtaaaaagaacattctctctcagattgtggacatagacggaatctgggaagggactcgagactttgcggcctgtaatcacttaagaagctacaaatattacactgatagcatcgtcaaccctgatggctttgctggattcccctgtgcctcttacaacgtcttcactgcaaacaagtgtttcccttgtccaagtggaggctgcccacagatgggtcactatgctgatagatatcctgggaaaacaaatgatgtgggccagaaattttatctagacactggtgatgccagtaattttgcacgttggaggtataaggtatctgtcacactgtctggaaaaaaggttacaggacacatactagtttctttgttcggaaataaaggaaactctaagcagtatgaaattttcaagggcactctcaaaccagatagtactcattccaatgaatttgactcagatgtggatgttggggacttgcagatggttaaatttatttggtataacaatgtgatcaacccaactttacctagagtgggagcatccaagattatagtggagacaaatgttggaaaacagttcaacttctgtagtccagaaaccgtcagggaggaagttctgctcaccctcacaccgtgttag

SEQ ID NO: 36 - DNA, 1536 bp, Homo sapiens
atgaagttctttctgttgcttttcaccattgggttctgctgggctcagtattccccaaatacacaacaaggacggacatctattgttcatctgtttgaatggcgatgggttgatattgctcttgaatgtgagcgatatttagctccgaagggatttggaggggttcaggtctctccaccaaatgaaaatgttgcaatttacaaccctttcagaccttggtgggaaagataccaaccagttagctataaattatgcacaagatctggaaatgaagatgaatttagaaacatggtgactagatgtaacaatgttggggttcgtatttatgtggatgctgtaattaatcatatgtgtggtaacgctgtgagtgcaggaacaagcagtacctgtggaagttacttcaaccctggaagtagggactttccagcagtcccatattctggatgggatttcaatgatggtaaatgtaaaactggaagtggagatatcgagaactacaatgatgctactcaggtcagagattgtcgtctgactggtcttcttgatcttgcactggagaaggattacgtgcgttctaagattgccgaatatatgaaccatctcattgacattggtgttgcagggttcagacttgatgcttccaagcacatgtggcctggagacataaaggcaattttggacaaactgcataatctaaacagtaactggttccctgcaggaagtaaacctttcatttaccaggaggtaattgatctgggtggtgagccaattaaaagcagtgactactttggtaatggccgggtgacagaattcaagtatggtgcaaaactcggcacagttattcgcaagtggaatggagagaagatgtcttacttaaagaactggggagaaggttggggtttcgtaccttctgacagagcgcttgtctttgtggataaccatgacaatcaacgaggacatggggctggaggagcctctattcttaccttctgggatgctaggctgtacaaaatggcagttggatttatgcttgctcatccttacggatttacacgagtaatgtcaagctaccgttggccaagacagtttcaaaatggaaacgatgttaatgattgggttgggccaccaaataataatggagtaattaaagaagttactattaatccagacactacttgtggcaatgactgggtctgtgaacatcgatggcgccaaataaggaacatggttattttccgcaatgtagtggatggccagccttttacaaattggtatgataatgggagcaaccaagtggcttttgggagaggaaacagaggattcattgttttcaacaatgatgactggtcattttctttaactttgcaaactggtcttcctgctggcacatactgtgatgtcatttctggagataaaattaatggcaattgcacaggcattaaaatttacgtttctgatgatggcaaagctcatttttctattagtaactctgctgaagatccatttattgcaattcatgctgaatctaaattgtaa

SEQ ID NO: 37 - DNA, 1536 bp, Homo sapiens
atgaagttctttctgttgcttttcaccattgggttctgctgggctcagtattccccaaatacacaacaaggacggacatctattgttcatctgtttgaatggcgatgggttgatattgctcttgaatgtgagcgatatttagctcccaagggatttggaggggttcaggtctctccaccaaatgaaaatgttgcaattcacaaccctttcagaccttggtgggaaagataccaaccagttagctataaattatgcacaagatctggaaatgaagatgaatttagaaacatggtgactagatgtaacaatgttggggttcgtatttatgtggatgctgtaattaatcatatgtctggtaatgctgtgagtgcaggaacaagcagtacctgtggaagttacttcaaccctggaagtagggactttccagcagtcccatattctggatgggattttaatgatggtaaatgtaaaactggaagtggagatatcgagaactacaatgatgctactcaggtcagagattgtcgtctggttggtcttcttgatcttgcactggagaaagattatgtgcgttccaagattgccgaatatatgaatcatctcattgacattggtgttgcagggttcagacttgatgcttccaagcacatgtggcctggagacataaaggcaattttggacaaactgcataatctaaacagtaactggttccctgcaggaagtaaacctttcatttaccaggaggtaattgatctgggtggtgagccaattaaaagcagtgactactttggaaatggccgggtgacagaattcaagtatggtgcaaaactcggcacagttattcgcaagtggaatggagagaagatgtcttacctaaagaactggggagaaggttggggtttcatgccttctgacagagcacttgtctttgtggataaccatgacaatcaacgaggacatggggctggaggagcctctattcttaccttctgggatgctaggctgtataaaatggcagttggatttatgcttgctcatccttatggttttacacgagtaatgtcaagctaccgttggccaagacagtttcaaaatggaaacgatgttaatgattgggttgggccaccaaataataatggagtaattaaagaagttactattaatccagacactacttgtggcaatgactgggtctgtgaacatcgatggcgccaaataaggaacatggttaatttccgcaatgtagtggatggccagccttttacaaactggtatgataatgggagcaaccaagtggcttttgggagaggaaacagaggattcattgttttcaacaatgatgactggacattttctttaactttgcaaactggtcttcctgctggcacatactgtgatgtcatttctggagataaaattaatggcaattgcacaggcattaaaatctacgtttctgacgatggcaaagctcatttttctattagtaactctgctgaggatccatttattgcaattcatgctgaatctaaattataa

SEQ ID NO: 38 - DNA, 1197 bp, Homo sapiens
atgtggctgcttttaacaatggcaagtttgatatctgtactggggactacacatggtttgtttggaaaattacatcctggaagccctgaagtgactatgaacattagtcagatgattacttattggggatacccaaatgaagaatatgaagttgtgactgaagatggttatattcttgaagtcaatagaattccttatgggaagaaaaattcagggaatacaggccagagacctgttgtgtttttgcagcatggtttgcttgcatcagccacaaactggatttccaacctgccgaacaacagccttgccttcattctggcagatgctggttatgatgtgtggctgggcaacagcagaggaaacacctgggccagaagaaacttgtactattcaccagattcagttgaattctgggctttcagctttgatgaaatggctaaatatgaccttccagccacaatcgacttcattgtaaagaaaactggacagaagcagctacactatgttggccattcccagggcaccaccattggttttattgccttttccaccaatcccagcctggctaaaagaatcaaaaccttctatgctctagctcctgttgccactgtgaagtatacaaaaagccttataaacaaacttagatttgttcctcaatccctcttcaagtttatatttggtgacaaaatattctacccacacaacttctttgatcaatttcttgctactgaagtgtgctcccgtgagatgctgaatctcctttgcagcaatgccttatttataatttgtggatttgacagtaagaactttaacacgagtcgcttggatgtgtatctatcacataatccagcaggaacttctgttcaaaacatgttccattggacccaggctgttaagtctgggaaattccaagcttatgactggggaagcccagttcagaataggatgcactatgatcagtcccaacctccctactacaatgtgacagccatgaatgtaccaattgcagtgtggaacggtggcaaggacctgttggctgacccccaagatgttggccttttgcttccaaaactccccaatcttatttaccacaaggagattcctttttacaatcacttggactttatctgggcaatggatgcccctcaagaagtttacaatgacattgtttctatgatatcagaagataaaaagtag

SEQ ID NO: 39 - DNA, 1830 bp, Homo sapiens
atgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataa

SEQ ID NO: 40 - DNA, 990 bp, Homo sapiens
gcaagcttcaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 41 - DNA, 981 bp, Homo sapiens
gcaagcttcaagggcccatcggtcttccccctggtgccctgctccaggagcacctccgagagcacagccgccctgggctgcctggtcaaggactacttccccgaaccggtgacggtgtcgtggaactcatgcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagggtcctcaccgtcctgcaccaggactggctgaacggtaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggaggacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaa

SEQ ID NO: 42 - DNA, 349 bp, Homo sapiens
atggagtttgggctgagctggctttttcttgtggctattttaaaaggtgtccagtgtgaggtgcagctgttggagtctgggggaggcttggtacagcctggggggtccctgagactctcctgtgcagcctctggattcacctttagcagctatgccatgagctgggtccgccagtctccagggaaggggctacagtgggtctcagctattagtggtagtggtattagcacatactacgcagactccgtgaggggccggttcaccatctccagagacaattccaagaacacgctgtatctgcaaatgagcagcctgagccgaggacacggccgtatattactgtgcgaaa

SEQ ID NO: 43 - DNA, 711 bp, Homo sapiens
atggacatgagggtccccgctcagctcctggggctcctgctgctctggctcccaggtgccagatgtgtcatctggatgacccagtctccatccttactctctgcatctacgggagacagagtcacaatcagttgtcggatgagtcagggcattagcaattatttagcctggtatcagcaaaaaccagggaaagcccctgacctcctgatctatgctgcatccactttgcaaagtggggtcccatcaaggttcagtggcagtggatctgggacagatttcattctcaccatcagccgcctgcagtctgaagattttgcaatttattactgtcaacagtattatagtttcccattcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 44 - DNA, 558 bp, Gaussia princeps
atgggagtgaaagttctttttgcccttatttgtattgctgtggccgaggccaaaccaactgaaaacaatgaagatttcaacattgtagctgtagctagcaactttgctacaacggatctcgatgctgaccgtggtaaattgcccggaaaaaaattaccacttgaggtactcaaagaaatggaagccaatgctaggaaagctggctgcactaggggatgtctgatatgcctgtcacacatcaagtgtacacccaaaatgaagaagtttatcccaggaagatgccacacctatgaaggagacaaagaaagtgcacagggaggaataggagaggctattgttgacattcctgaaattcctgggtttaaggatttggaacccatggaacaattcattgcacaagttgacctatgtgtagactgcacaactggatgcctcaaaggtcttgccaatgtgcaatgttctgatttactcaagaaatggctgccacaaagatgtgcaacttttgctagcaaaattcaaggccaagtggacaaaataaagggtgccggtggtgattaa

SEQ ID NO: 45 - DNA, 507 bp, Gaussia princeps
atgccaactgaaaacaatgaagatttcaacattgtagctgtagctagcaactttgctacaacggatctcgatgctgaccgtggtaaattgcccggaaaaaaattaccacttgaggtactcaaagaaatggaagccaatgctaggaaagctggctgcactaggggatgtctgatatgcctgtcacacatcaagtgtacacccaaaatgaagaagtttatcccaggaagatgccacacctatgaaggagacaaagaaagtgcacagggaggaataggagaggctattgttgacattcctgaaattcctgggtttaaggatttggaacccatggaacaattcattgcacaagttgacctatgtgtagactgcacaactggatgcctcaaaggtcttgccaatgtgcaatgttctgatttactcaagaaatggctgccacaaagatgtgcaacttttgctagcaaaattcaaggccaagtggacaaaataaagggtgccggtggtgattaa

SEQ ID NO: 46 - DNA, 19 bp, Artificial sequence
  Primer
cattgtagctgtagctagc

SEQ ID NO: 47 - DNA, 17 bp, Artificial sequence
  Primer
ttaatcaccaccggcac

SEQ ID NO: 48 - DNA, 579 bp, Mus musculus
atgggggtgcccgaacgtcccaccctgctgcttttactctccttgctactgattcctctgggcctcccagtcctctgtgctcccccacgcctcatctgcgacagtcgagttctggagaggtacatcttagaggccaaggaggcagaaaatgtcacgatgggttgtgcagaaggtcccagactgagtgaaaatattacagtcccagataccaaagtcaacttctatgcttggaaaagaatggaggtggaagaacaggccatagaagtttggcaaggcctgtccctgctctcagaagccatcctgcaggcccaggccctgctagccaattcctcccagccaccagagacccttcagcttcatatagacaaagccatcagtggtctacgtagcctcacttcactgcttcgggtactgggagctcagaaggaattgatgtcgcctccagataccaccccacctgctccactccgaacactcacagtggatactttctgcaagctcttccgggtctacgccaacttcctccgggggaaactgaagctgtacacgggagaggtctgcaggagaggggacaggtga

SEQ ID NO: 49 - DNA, 504 bp, Mus musculus
atggctcccccacgcctcatctgcgacagtcgagttctggagaggtacatcttagaggccaaggaggcagaaaatgtcacgatgggttgtgcagaaggtcccagactgagtgaaaatattacagtcccagataccaaagtcaacttctatgcttggaaaagaatggaggtggaagaacaggccatagaagtttggcaaggcctgtccctgctctcagaagccatcctgcaggcccaggccctgctagccaattcctcccagccaccagagacccttcagcttcatatagacaaagccatcagtggtctacgtagcctcacttcactgcttcgggtactgggagctcagaaggaattgatgtcgcctccagataccaccccacctgctccactccgaacactcacagtggatactttctgcaagctcttccgggtctacgccaacttcctccgggggaaactgaagctgtacacgggagaggtctgcaggagaggggacaggtga

SEQ ID NO: 50 - DNA, 21 bp, Artificial sequence
  Primer
cacgatgggttgtgcagaagg

SEQ ID NO: 51 - DNA, 22 bp, Artificial sequence
  Primer
cgaagcagtgaagtgaggctac

SEQ ID NO: 52 - DNA, 405 bp, Homo sapiens
atggcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 53 - DNA, 768 bp, Artificial sequence
  chimeric enhanced GFP
atgggagtgaaagttctttttgcccttatttgtattgctgtggccgaggccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 54 - DNA, 786 bp, Artificial sequence
  chimeric enhanced GFP with an Histidin Tag
atgggagtgaaagttctttttgcccttatttgtattgctgtggccgaggccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcaccaccatcaccaccattaa